(19) United States
(12) Patent Application Publication
(10) Pub. No.: US 2018/0028658 A1
(43) Pub. Date: Feb. 1, 2018

(54) ANTIBODY-DRUG CONJUGATES FOR REDUCING THE LATENT HIV RESERVOIR

(71) Applicant: New York Blood Center, Inc., New York, NY (US)

(72) Inventors: Asim Kumar Debnath, Fort Lee, NJ (US); Hongtao Zhang, Mt. Vernon, NY (US)

(21) Appl. No.: 15/551,130

(22) PCT Filed: Feb. 16, 2016
(86) PCT No.: PCT/US16/18115

§ 371 Date: Aug. 15, 2017

Related U.S. Application Data
(60) Provisional application No. 62/116,782, filed on Feb. 16, 2015.

(51) Int. Cl. A61K 39/42; C07K 16/18; A61K 45/06

(52) U.S. Cl. CPC A61K 39/42; A61K 45/06; C07K 16/18

(57) ABSTRACT

Disclosed herein are antibody-drug conjugates having the general formula A-L-D, wherein A represents a neutralizing antibody specific for human immunodeficiency virus (HIV), L represents a linker, and D represents a cytotoxic moiety, pharmaceutical compositions comprising the antibody-drug conjugates, and methods of treating HIV with the antibody-drug conjugates after activation of latent reservoir cells.

### FIELD

The present disclosure relates to the field of therapeutic agents and treatment strategies for human immunodeficiency virus infection.

### BACKGROUND

More than thirty United States Food and Drug Administration (FDA)-approved antiretroviral (ARV) drugs make considerable impact in treating millions of patients infected with the Human Immunodeficiency Virus (HIV). These drugs greatly decrease the mortality of HIV-infected individuals and successfully suppress the plasma virus RNA load to below the limit of the detection (50 copies of HIV RNA/ml plasma). Unfortunately, even after long-term antiretroviral therapy, the virus persists in T cells and patient who stop drug therapy exhibit a rebound of HIV virus. The residual viruses primarily arise from about 10⁶ to 10⁷ slow-replicating latent memory T cells (latent reservoir) which remain dormant and can be reactivated. Eliminating viruses from the latent reservoir is the stumbling block in developing effective strategies to treat HIV patients.

One strategy, the “shock and kill” approach, showed potential. In this approach, the latent infected cells are shocked by agents that reactivate these cells which leads to viral RNA synthesis, production of viral proteins, and eventually release of replication competent virus particles. These virus particles are then killed by natural means such as immune response or cytopathogenicity or by drugs, monoclonal antibodies, etc. Histone deacetylase inhibitors (HDACs) and protein kinase C inhibitors have been identified and showed promise to reactivate latently infected cells. Varinostat, one of the HDAC inhibitors, disrupted HIV latency in the resting CD4+ T cells of patients whose viremia was fully controlled by ARV treatment. These reagents were expected to eliminate latent HIV reservoirs. However, other studies showed that these drugs were not capable of eliminating the latent viral reservoir cells. A very similar observation has been reported recently for panobinostat, a HDAC inhibitor. Therefore, finding alternate approaches to eliminate or reduce virus reservoirs is urgently needed.

### SUMMARY

Disclosed herein are antibody-drug conjugates for the treatment of HIV infection, therein the antibody-drug conjugate comprising the general formula:

A-L-D

wherein A represents a broadly neutralizing antibody specific for human immunodeficiency virus (HIV), L represents a linker, and D represents a cytotoxic moiety.

In certain embodiments, the antibody is a mouse antibody, a human antibody, a chimeric antibody, a humanized antibody, or an antibody fragment. In some embodiments, the antibody is a Fab, Fab′, F(ab′)₂, and Fv fragments, a diabody, a triabody, a tetrabody, a linear antibody, a single-chain antibody molecule, a scFv, or a scFv-Fc antibody fragment. In some embodiments, the antibody is NIH45-46 G54W.

In certain embodiments, the linker is a cleavable linker or a non-cleavable linker. In some embodiments, the cleavable linker is a dipeptide linker. In some embodiments, the dipeptide linker is valine-citrulline, histidine-valine, aspartic acid-valine, isoleucine-valine, asparagine-valine, tyrosine-valine, E-N-trimethyllysine-proline, isoleucine-proline, tyrosine-aspartic acid, norvaline-aspartic acid, phenylglycine-lysine, methionine-lysine, or asparagine-lysine. In some embodiments, the dipeptide linker is valine-citrulline. In other embodiments, the non-cleavable linker is succinimidyl trans-4-(maleimidylmethyl)cyclohexane-1-carboxylate (SMMC).

In certain embodiments, the cytotoxic moiety is an auristatin, a maytanasine, a calicheasmicin, a duocarymycin, a PDB dimer, or an amanitin. In some embodiments, the auristatin is MMAE (N-methylvaline-valine-dolaisoleuine-dolaproine-norephedrine), MMAF (N-methylvaline-valine-dolaisoleuine-dolaproine-phenylalanine), or AF (N,N-dimethylvaline-valine-dolaisoleuine-dolaproine-phenylalanine). In some embodiments, the maytanasine is DM1 or DM4.

In some embodiments, the antibody-drug conjugate is produced as a fusion protein and comprises an antibody, a dipeptide linker, and an auristatin in that order. In some embodiments, the antibody is NIH45-46 G54W, the linker is valine-citrulline, and the auristatin is MMAE.

Also disclosed herein is an antibody-drug conjugate comprising a broadly neutralizing HIV-specific antibody, N-methylvaline-valine-dolaisoleuine-dolaproine-norephedrine, and a valine-citrulline linker.

Further provided are pharmaceutical compositions comprising an antibody-drug conjugate disclosed herein, and a pharmaceutically acceptable excipient.

Also provided are methods of treating a subject infected with a human immunodeficiency virus comprising administering an antibody-drug conjugate disclosed herein to the subject. In some embodiments, the method further comprises first activating latent HIV-infected cell from the latent reservoir with a latency-reversing agent.

Also provided are methods of reducing the latent reservoir of HIV in a subject infected with HIV comprising administering an antibody-drug conjugate disclosed herein to the subject. In some embodiments, the method further comprises first activating latent HIV-infected cell from the latent reservoir with a latency-reversing agent.

In some embodiments, the latency-reversing agent is a histone deacetylase (HDAC) inhibitor, a protein kinase C (PKC) activator, a histone methyltransferase (HMT), a DNA methyltransferase inhibitor (DNMTI), a bromodomain and extra terminal (BET) domain-containing protein inhibitor, a toll-like receptor (TLR) agonist, or a Smac (second mitochondrial-derived activator of caspases) mimetic, and combinations thereof.

### BRIEF DESCRIPTION OF THE DRAWINGS

FIG. 1 depicts antibody screening by flow cytometry. Thirty-four human-derived monoclonal antibodies were obtained from NIH AIDS Reagents Program and used to detect 293T cell surface Env. All available antibodies were used as a primary antibody at 10 μg/ml and further probed by FITC-conjugated second antibody. These primary antibodies are classified as antibody directed to CD4-induced conformation on gp120 (CD4-i), glycan-dependent epitope (glycan), CD4-binding sites on gp120 (CD4bs), adjacent to CD4 binding sites (adjCD4bs), V1, V2/V3 loop of gp120 (v1v2v3) and gp41. The antibody detection rates were compared using a T test. The mean values are shown as black bars.

FIG. 2A-H depicts the HIV Env expressed on the 293T cell surfaces by flow cytometry using monoclonal antibodies (mAbs) 17b (FIG. 2A), NIH45-46 G54W (FIG. 2B), PG9 (FIG. 2C), PG16 (FIG. 2d), b12 (FIG. 2E), 2G12 (FIG. 2F), VRC03 (FIG. 2G), and VRC01 (FIG. 2H) and fluorescein isothiocyanate (FITC) labeled second antibodies.

FIG. 3A-C depicts the binding affinity of antibodies VRC01, NIH45-46, and NIH45-46 G54W for the cleavable version gp140 trimer (BG505 SOSIP.664) (FIG. 3A), the non-cleavable version of gp140 trimer (SF162) (FIG. 3B), and the affinity of control antibodies 17b, 19b, D50, HJ16, 2G12, and b12 to the cleavable version gp140 trimer (BG505 SOSIP.664) (FIG. 3C). PBS was used as a control.

FIG. 4A-B depicts the binding affinity of antibodies VRC01, NIH45-46, and NIH45-46 G54W (FIG. 4A) and control antibodies 17b, 19b, D50, 2G12 and b12 (FIG. 4B) for the HIV-1 gp120 monomer. PBS was used as a control.

FIG. 5A-B depicts an estimation of binding affinity of mAbs to HIV-1 gp41 ectodomain by ELISA. FIG. 5A depicts the binding affinity of NIH45-46 and NIH45-46 G54W to gp41-ectomain. The control antibodies are 17b, 2F5, 4E10, and VRC01. FIG. 5B depicts the binding affinity of NIH45-46 G54W to gp41 from HIV-1Yu2, BaL and HXB2.

FIG. 6A-D depicts a kinetics analysis of NIH45-46 G54W with HIV-1 gp41 by surface plasmon resonance (SPR) (FIG. 6B). gp41 protein was immobilized on CM5 chips and different concentrations of antibody were passed over the chip and the response measured in resonance units (RU). The injection time was 2 min and the disassociation time was 5 min. Monoclonal antibody 17b (FIG. 6A) was used as negative control and mAbs 4E10 (FIG. 6C) and 10E8 (FIG. 6D) were used as positive controls.

FIG. 7A-D depicts a kinetics analysis between antibodies and HIV-1 gp140 trimer (BG505 SOSIP.646) or HIV-1 gp120 monomer by SPR. Either gp140 trimer (BG505 SOSIP.646) or gp120 monomer was immobilized on CM5 chips and different concentrations of antibody were passed over the chip and the response measured in RU. The injection time was 2 min and the disassociation time was 5 min. FIG. 7A depicts antibody 10E8 vs. gp140 trimer; FIG. 7B depicts antibody NIH45-46 G54W vs. gp140 trimer; FIG. 7C depicts antibody 17b vs. gp120 monomer; and FIG. 7D depicts antibody NIH45-46 vs. gp120 monomer.

FIG. 8A-C depicts flow cytometric detection of CV-1 cell surface gp41 by antibody NIH45-46 G54W. The C-terminal part of gp41 was expressed using recombinant vaccinia virus (vP1340), NIH45-46 G54W was used as the primary antibody at 10 μg/ml and further probed by FITC-conjugated second antibody and flow cytometry analysis (FIG. 8C). 17b (FIG. 8A) and 10E8 (FIG. 8C) were used as control antibodies.

FIG. 9A-B depicts titration of antibody recognition of HIV Env expressed on activated ACH-2 cell surface by flow cytometry. FIG. 9A depicts NIH45-46 G54W used as primary antibody to detect ACH-2 cell surface Env by flow cytometry after 3 days of reactivation in presence of 10 ng/ml of TNF-α. The starting concentration of primary antibody was 20 μg/ml and further serially diluted at 1:3 for another 6 dilutions. Then the cell surface Env proteins were probed by FITC-conjugated second antibody and subjected to FACS analysis. FIG. 9B depicts the dose (log)-signal plot of NIH45-46 G54W by GRAPHPAD PRISM™ to calculate the half-maximal dose (EC₅₀=5.2 μg/ml).

FIG. 10 depicts the measurement of the internalization of antibody NIH45-46 G54W by flow cytometry. After 3 days of activation with 10 ng/ml TNF-α, ACH-2 cells were incubated with 8 μg/ml FITC-labeled NIH45-46 G54W for different intervals (0, 15 min, 1 h or 4 h) at 37° C., followed by FACS analysis. FITC-NIH45-46 G54W internalization occurred in a time-dependent manner at 37° C.

FIG. 11A-F depicts measurement of internalization of antibody NIH45-46 G54W in Env-expressing 293T cells by confocal microscopy. FITC-NIH45-46 G54W internalization is dependent on the cell surface expression of HIV-1 Env. Representative confocal microscopy images of Env-expressing-vector-transfected 293T cells incubated for 4 h at 37° C. with FITC-conjugated antibody. FIG. 11A depicts a differential interference contrast (DIC) image of cells with FITC-conjugated control human antibody; FIG. 11B depicts a FITC fluorescent image of the same cells with FITC-conjugated control human antibody; and FIG. 11C depicts an overlay of DIC and FITC fluorescent images. FIG. 11D depicts a DIC image of cells with FITC-conjugated NIH45-46 G54W; FIG. 11E depicts a FITC fluorescent image of the same cells with FITC-conjugated NIH45-46 G54W; and FIG. 11F depicts an overlay of DIC and FITC fluorescent images.

FIG. 12 depicts in vitro killing of activated ACH-2 cells by vc-MMAE-conjugated NIH45-46 G54W. Activated ACH-2 and A3.01 cells were plated at 5×10³ cells/well and were exposed to a 1:3 diluted culture medium containing vc-MMAE-conjugated NIH45-46 G54W or vc-MMAE-conjugated anti-HER2. After 3 days of incubation, cells were assessed for killing by a CELLTITER-BLUE® cell viability assay. The florescence density was plotted against the log of antibody-drug conjugate (ADC) concentration. Results for each study are the average of triplicate determinations. Error bars indicate standard deviation (SD).

### DETAILED DESCRIPTION

Antibody-based therapies for cancer have come a long way after about twenty years of research and several antibodies have been approved by the U.S. Food and Drug Administration (FDA) and European regulatory authorities. Of particular interest is the rapid development of antibody-drug conjugate (ADC)-based therapy for tumor antigen-directed killing of tumor cells. Currently two such ADCs, ADCETRIS® (brentuximab vedotin) by Seattle Genetics and KADCYLA® (ado-trasuzumab emtansine) by Roche, are approved by the FDA and used in the treatment of cancer.

The Human Immunodeficiency Virus (HIV) is able to remain a chronic, life-long infection due to its ability to establish a latent reservoir of infected cells. The HIV particles remain invisible to the body's immune defenses and are not sensitive to anti-HIV drugs.

The latent reservoir for HIV-1 is a population of long-lived resting memory CD4+ T cells with integrated HIV-1 DNA. Latent reservoirs of HIV are located throughout the body, including the brain, lymphoid tissue, bone marrow, and the genital tract. After establishment during acute infection, it increases to 10⁵ to 10⁷ cells and then remains stable. As only replicating virus is targeted by antiretroviral therapy, latently infected cells persist even after years of effective treatment. Cellular activation leads to virus production and, if treatment is interrupted, viremia rebounds within weeks.

Quite a few highly potent anti-HIV bNAbs have been developed and some show potential in suppressing HIV in latent reservoirs. A combination of bNAbs and latency-reversing agents (activators of HIV-infected latent cells) can diminish the latent reservoir in hu-mice to a level without viral rebound after discontinuing the antibody therapy. The mechanism of the suppression of HIV infectivity was due to the ability of bNAbs to engage with the immune system through Fc receptor binding. Furthermore, several highly potent bNAbs such as PGT121, VRC01 and VRC03 potently inhibited HIV induced from infected patients whose plasma viremia is controlled with ART.

A number of these bNAbs are potent binders of envelope surface glycoproteins of HIV and broadly neutralize large number of clinical isolates in a single-cycle infectivity assay. The envelope glycoprotein antigens are not present on uninfected human cells. However upon induction, the viral envelope glycoproteins are expressed on the latently infected cell surface which can be detected by the bNAbs. Thus these bNAbs are useful in the construction of ADCs. Exemplary bNAbs are presented in Table1.

In order to produce a drug conjugate, an antibody is conjugated to a cytotoxic agent via a linker which can allow the cytotoxic agent to specifically act at the target site. Thus, disclosed herein are ADC according to the formula A-L-D, wherein A is a broadly neutralizing anti-HIV monoclonal antibody, L is a peptide linker, and D is a cytotoxic agent.

The term “antibody” is used herein in the broadest sense and specifically covers intact monoclonal antibodies, polyclonal antibodies, monospecific antibodies, multispecific antibodies (e.g., bispecific antibodies), and antibody fragments that exhibit the desired biological activity. An intact antibody primarily has two regions: a variable region and a constant region. The variable region binds to and interacts with a target antigen. The variable region includes a complementary determining region (CDR) that recognizes and binds to a specific binding site on a particular antigen. The constant region may be recognized by and interact with the immune system. An antibody can be of any type or class (e.g., IgG, IgE, IgM, IgD, and IgA) or subclass (e.g., IgGI₅ IgG2, IgG3, IgG4, IgAI and IgA2). An antibody or bNAb can be, for example, murine, human, humanized, or chimeric.

The term “broadly neutralizing antibody” refers to antibodies which neutralize multiple HIV viral strains. In contrast, non-bNAbs are specific for individual viral strains. Most antibodies work by binding to an antigen on a cell's surface, signaling to a white blood cell that this antigen has been targeted, after which the antigen and cell are processed and consequently destroyed. The difference between neutralizing antibodies and binding antibodies is that neutralizing antibodies neutralize the biological effects of the antigen, whereas binding antibodies flag antigens. This difference is what gives neutralizing antibodies the ability to fight viruses which attack the immune system, since they can neutralize function without a need for white blood cells.

The terms “specifically binds” and “specific binding” refer to an antibody binding to a predetermined antigen. Typically, the antibody binds with an affinity of at least about 1×10⁻⁷ M, and binds to the predetermined antigen with an affinity that is at least two-fold greater than its affinity for binding to a non-specific antigen (e.g., BSA, casein) other than the predetermined antigen or a closely-related antigen.

The term “monoclonal antibody” as used herein refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. The modifier “monoclonal” indicates the character of the antibody as being obtained from a substantially homogeneous population of antibodies, and is not to be construed as requiring production of the antibody by any particular method. The term monoclonal antibodies specifically includes murine, human, chimeric, and humanized antibodies. A chimeric antibody refers to an antibody in which a portion of the heavy and/or light chain is identical to or homologous to the corresponding sequence of antibodies derived from a particular species or belonging to a particular antibody class or subclass, while the remainder of the chain(s) is identical to or homologous to the corresponding sequences of antibodies derived from another species or belonging to another antibody class or subclass, as well as fragments of such antibodies, so long as they exhibit the desired biological activity. In one embodiment, a chimeric antibody comprises variable heavy and variable light chains from a rodent antibody and constant light and constant heavy chains from a human antibody

An “intact antibody” is one which comprises an antigen-binding variable region as well as a light chain constant domain (C_{L}) and heavy chain constant domains, C_{H}1, C_{H}2, C_{H}3 and C_{H}4, as appropriate for the antibody class. The constant domains may be native sequence constant domains (e.g., human native sequence constant domains) or amino acid sequence variants thereof.

An intact antibody may have one or more “effector functions”, which refer to those biological activities attributable to the Fc region (e.g., a native sequence Fc region or amino acid sequence variant Fc region) of an antibody. Examples of antibody effector functions include complement dependent cytotoxicity, antibody-dependent cell-mediated cytotoxicity (ADCC), and antibody-dependent cell-mediated phagocytosis.

An “antibody fragment” comprises a portion of an intact antibody, preferably comprising the antigen-binding or variable region thereof. Examples of antibody fragments include Fab, Fab′, F(ab′)₂, and Fv fragments, diabodies, triabodies, tetrabodies, linear antibodies, single-chain antibody molecules, scFv, scFv-Fc, multispecific antibody fragments formed from antibody fragment(s), a fragment(s) produced by a Fab expression library, or an epitope-binding fragments of any of the above which immunospecifically bind to a target antigen (e.g., an HIV antigen).

The term “variable” in the context of an antibody refers to certain portions of the variable domains of the antibody that differ extensively in sequence and are used in the binding and specificity of each particular antibody for its particular antigen. This variability is concentrated in three segments called “hypervariable regions” in the light chain and the heavy chain variable domains. The more highly conserved portions of variable domains are called the framework regions (FRs). The variable domains of native heavy and light chains each comprise four FRs connected by three hypervariable regions.

The term “hypervariable region” when used herein refers to the amino acid residues of an antibody which are responsible for antigen-binding. The hypervariable region generally comprises amino acid residues from a “complementarity determining region” or “CDR” (e.g., residues 24-34 (L1), 50-56 (L2), and 89-97 (L3) in the light chain variable domain and 31-35 (H1), 50-65 (H2), and 95-102 (H3) in the heavy chain variable domain) and/or those residues from a “hypervariable loop” (e.g., residues 26-32 (L1), 50-52 (L2), and 91-96 (L3) in the light chain variable domain and 26-32 (H1), 53-55 (H2), and 96-101 (H3) in the heavy chain variable domain). FR residues are those variable domain residues other than the hypervariable region residues as herein defined.

A “single-chain Fv” or “scFv” antibody fragment comprises the V_{R} and V_{L} domains of an antibody, wherein these domains are present in a single polypeptide chain. Typically, the Fv polypeptide further comprises a polypeptide linker between the V_{H} and V_{L} domains which enables the scFv to form the desired structure for antigen binding.

The term “diabody” refers to small antibody fragments with two antigen-binding sites, which fragments comprise a variable heavy domain (V_{H}) connected to a variable light domain (V_{L}) in the same polypeptide chain. By using a linker that is too short to allow pairing between the two domains on the same chain, the domains are forced to pair with the complementary domains of another chain and create two antigen-binding sites.

“Humanized” forms of non-human (e.g., rodent) antibodies are antibodies that contain minimal sequences derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and effector functions. In some instances, FR residues of the human immunoglobulin are replaced by corresponding non-human residues. Furthermore, humanized antibodies may comprise residues that are not found in the recipient antibody or in the donor antibody; these modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin.

The availability of better and more stable linkers has changed the function of the chemical bond. The type of linker, cleavable or noncleavable, lends specific properties to the cytotoxic (cell killing) drug. For example, a non-cleavable linker keeps the drug within the cell. As a result, the entire antibody, linker and cytotoxic (anti-HIV) agent enter the targeted HIV-infected cell where the antibody is degraded to the level of an amino acid. The resulting complex—amino acid, linker and cytotoxic agent—now becomes the active drug. In contrast, cleavable linkers are catalyzed by enzymes in the cell where cytotoxic agent is released. The difference is that the cytotoxic payload delivered via a cleavable linker can escape from the targeted cell and, in a process called “bystander killing,” attack neighboring cells. If the neighboring cells are HIV-infected, this allows killing for potentially more than one target cell by the ADC.

Exemplary peptide cleavable linkers for the ADC include, but are not limited to, dipeptide linkers, such as valine-citrulline, histidine-valine, aspartic acid-valine, isoleucine-valine, asparagine-valine, tyrosine-valine, -ε-N-trimethyllysine-proline, isoleucine-proline, tyrosine-aspartic acid, norvaline-aspartic acid, phenylglycine-lysine, methionine-lysine, and asparagine-lysine and others as disclosed in WO2009117531 which is incorporated by reference herein for all it discloses regarding dipeptide linkers.

Exemplary cytotoxic drugs for use in ADCs may include, but are not limited to, auristatins, maytanasines (mertaserines, i.e., DM1, DM4, etc.), calicheasmicins (i.e., calicheamicin γ1, esperamicin, etc.), duocarymycins (i.e., duocarymycin A, duocarymycin B1, duocarymycin B2, duocarymycin C1, duocarymycin C2, duocarymycin D, duocarymycin SA, CC-1065, etc.), α-amanitin, doxorubicin, D6.5, pyrrolobenzodiazepines (PBDs), centanamycin (i.e., ML-970; indolecarboxamide), SN38 (i.e., irinotecan metabolite), and combinations thereof.

Auristatins are derivatives of dolastatin 10 and representative auristatins include MMAE (N-methylvaline-valine-dolaisoleuine-dolaproine-norephedrine), MMAF (N-methylvaline-valine-dolaisoleuine-dolaproine-phenylalanine), and AF (N,N-dimethylvaline-valine-dolaisoleuine-dolaproine-phenylalanine).

The auristatin cytotoxic agents may be prepared according to the general methods of: U.S. Pat. No. 5,635,483; U.S. Pat. No. 5,780,588; Pettit et al., 1989, J. Am. Chem. Soc. 111:5463-5465; Pettit et al, 1998, Anti-Cancer Drug Design 13:243-277; and Pettit et al 1996, J Chem. Soc. Perkin Trans. 1 5:859-863.

Typically, peptide-based cytotoxic agents may be prepared by forming a peptide bond between two or more amino acids and/or peptide fragments. Such peptide bonds can be prepared, for example, according to the liquid phase synthesis method (see E. Schroder and K. Lübke, “The Peptides”, volume 1, pp 76-136, 1965, Academic Press) that is well known in the field of peptide chemistry.

Additionally, the antibody may be linked to a small molecule cytotoxic agent using a cross-linking reagent. One exemplary non-cleavable cross-linking agent is SMCC, or succinimidyl trans-4-(maleimidylmethyl)cyclohexane-1-carboxylate, a heterobifunctional crosslinker which contains two reactive functional groups, a succinimide ester, and a maleimide. The succinimide group of SMCC reacts with the free amino group of a lysine residue in the anitbody and the maleimide moiety of SMCC links to a free sulfhydryl group, causing the cytotoxic agent to form a non-cleavable covalent bond between the antibody and cytotoxic agent. Each antibody molecule may be linked to from about zero to about eight cytotoxic agent molecules. In exemplary embodiments, each antibody molecule is linked to one, two, three, four, five, six, seven, or eight cytotoxic agent molecules.

In one exemplary embodiment, the ADC comprises the anti-HIV broadly neutralizing antibody (bNAb) NIH45-46 G54W linked to the cytotoxic agent, monomethyl auristin E (MMAE) through a valine-citrulline linker, and its successful use leads to the killing latently infected cells in vitro.

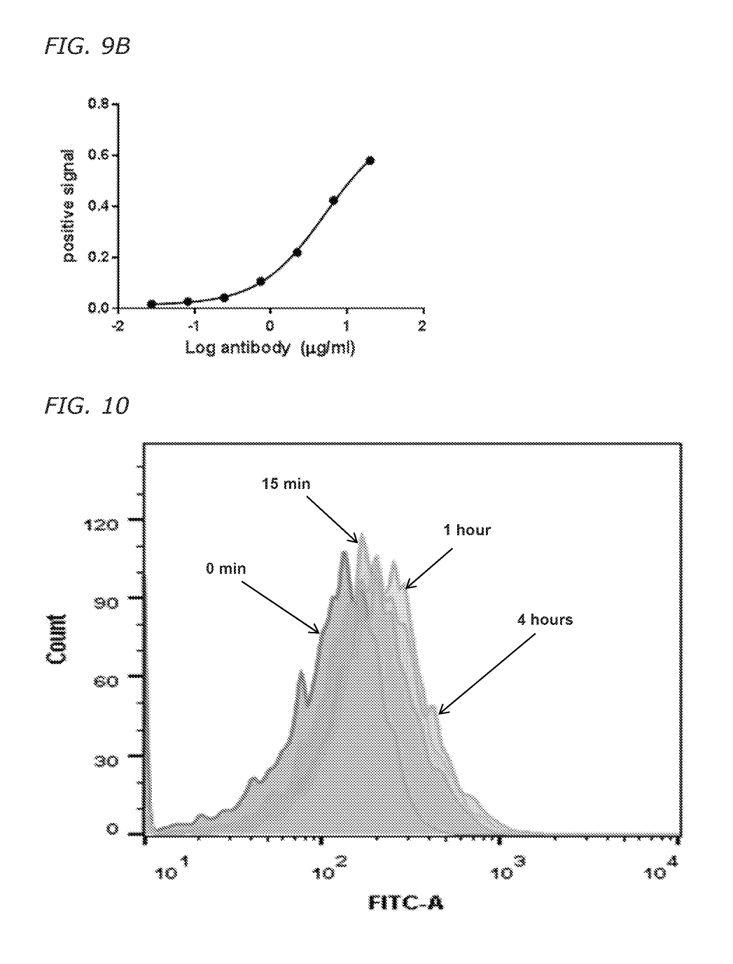

As demonstrated herein, NIH45-46 G54W is a good candidate bNAb for the preparation of an ADC due to its high affinity binding to gp120 envelope trimmer as well as gp41 trimer, and has low autoreactivity. Most significantly, NIH45-46 G54W showed a high level of recognition of the envelope expressed on the reactivated latently infected cells (FIG. 9A). Furthermore, FITC-conjugated NIH45-46 G54W can efficiently internalize (FIG. 10).

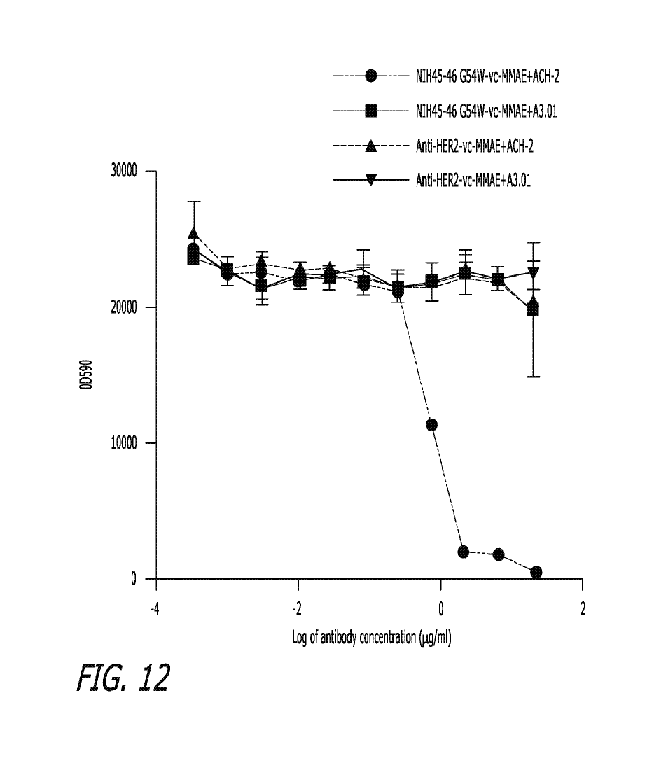

In one exemplary embodiment, valine-citrulline (vc) was selected as a linker and MMAE as the cytotoxic drug to prepare the ADC because these agents were previously determined to be optimal for an FDA-approved ADC drug, ADCETRIS® (brentuximab vedotin) and their pharmacokinetics and safety profiles were already validated. The vc-MMAE-N1H45-46 G54W (NYBC-HC-001) ADC showed potent (EC₅₀=850 ng/ml) and selective killing of the latently infected ACH-2 cells that express Env after reactivation with TNF-α, whereas a negative control ADC vc-MMAE-anti-HER2 showed no killing activity at all as seen in FIG. 12. Similarly, when 3.01 cells (the parental cells of ACH-2, which are not latently infected by HIV) were targeted, NYBC-HC-001 showed no cell killing at the highest dose tested indicating the cell killing property is HIV Env dependent.

Also disclosed herein are methods of treating HIV infection by administering to a subject in need thereof at least one latency-reversing agent (LRA) to activate or “awaken” HIV-infected cells from a latent state, and then administering an ADC disclosed herein to kill the activated HIV-infected cells.

Latency-reversing agents include, but are not limited to, histone deacetylase (HDAC) inhibitors, protein kinase C (PKC) activators, histone methyltransferases (HMT), DNA methyltransferase inhibitors (DNMTI), bromodomain and extra terminal (BET) domain-containing protein inhibitors, toll-like receptor (TLR) agonists, and Smac (second mitochondrial-derived activator of caspases) mimetics, and combinations thereof.

Exemplary HDAC inhibitors include, but are not limited to, vorinostat (SAHA), panobinostat (LBH589), romidepsin, and entinostat (MS-275).

Exemplary PKC activators include, but are not limited to, bryostatin-1, prostratin, ingenol-3-angelate (PEP005), and analogs thereof. Useful PKC activators, and analogs thereof, can be found in U.S. Pat. No. 6,624,189, U.S. Pat. No. 7,232,842, U.S. Pat. No. 7,256,286, U.S. Pat. No. 8,497,285, U.S. Pat. No. 8,536,378, U.S. Pat. No. 8,735,609, U.S. Pat. No. 8,816,122, US2007/0270485, and US2010/0280262, all of which are incorporated by reference herein for all they disclose regarding PKC activators.

Exemplary BET inhibitors include, but are not limited to, I-BET151 (GSK1210151A), I-BET726 (GSK1324726A), disulfiram, and JQ-1.

Exemplary Smac mimetics include, but are not limited to, LCL161, birinapant, SB1-0637142.

The term “therapeutically effective amount” refers to an amount of a drug effective to treat a disease or disorder in a mammal. In the case of HIV treatment, the therapeutically effective amount of the drug may reduce the number of infected cells, reduce the amount of infectious virus particles, reduce the latent reservoir, or otherwise give the mammal a better prognosis that had the mammal not been treated. In some embodiments, an “effective amount” of an HIV treatment disclosed herein, when administered in one or more doses to an individual having a virus infection, is effective to achieve about a 1.5-log, about a 2-log, about a 2.5-log, about a 3-log, about a 3.5-log, about a 4-log, about a 4.5-log, or about a 5-log reduction in viral titer in the serum of the individual, or any other value bound by these ranges.

Using the methods disclosed herein, the latent reservoir of HIV can be reduced by about 1 log, about 1.5 log, about 2 log, about 2.5 log, about 3 log, about 3.5 log, about 4 log, about 4.5 log, or about 5 log, or other values bound by these ranges. In certain embodiments, the HIV serum viral titer in a subject can be reduced to substantially zero, even after administration of a LRA.

In other embodiments, presently described is a pharmaceutical composition including an effective amount of an ADC and a pharmaceutically acceptable carrier or vehicle. The compositions are suitable for veterinary or human administration.

The present pharmaceutical compositions may be in any form that allows for the composition to be administered to a patient. For example, the composition can be in the form of a solid or liquid. Typical routes of administration include, without limitation, parenteral and oral. Parenteral administration includes subcutaneous injections, intravenous, intramuscular, or intrasternal injection or infusion techniques.

Pharmaceutical compositions can be formulated so as to allow an ADC to be bioavailable upon administration of the composition to a patient. Compositions can take the form of one or more dosage units, where, for example, a tablet can be a single dosage unit, and a container of an ADC in liquid form can hold a plurality of dosage units.

Materials used in preparing the pharmaceutical compositions can be non-toxic in the amounts used. It will be evident to those of ordinary skill in the art that the optimal dosage of the active ingredient(s) in the pharmaceutical composition will depend on a variety of factors. Relevant factors include, without limitation, the type of animal (e.g., human), the particular form of the ADC, the manner of administration, and the composition employed.

The pharmaceutically acceptable carrier or vehicle may be solid or particulate, so that the compositions are, for example, in tablet or powder form. The carrier(s) may be liquid. In addition, the carrier(s) may be particulate.

The composition may be in the form of a liquid, e.g., a solution, emulsion or suspension. In a composition for administration by injection, one or more of a surfactant, preservative, wetting agent, dispersing agent, suspending agent, buffer, stabilizer and isotonic agent may also be included.

The liquid compositions, whether they are solutions, suspensions or other like form, may also include one or more of the following: sterile diluents such as water for injection, saline solution, preferably physiological saline, Ringer's solution, isotonic sodium chloride, fixed oils such as synthetic mono or digylcerides which can serve as the solvent or suspending medium, polyethylene glycols, glycerin, cyclodextrin, propylene glycol, or other solvents; antibacterial agents such as benzyl alcohol or methyl paraben; antioxidants such as ascorbic acid or sodium bisulfite; chelating agents such as ethylenediaminetetraacetic acid (EDATA); buffers such as acetates, citrates, phosphates, or amino acids and agents for the adjustment of tonicity such as sodium chloride or dextrose. A parenteral composition may be enclosed in ampule, a disposable syringe, or a multiple-dose vial made of glass, plastic, or other material. Physiological saline is an exemplary carrier. An injectable composition is preferably sterile.

The amount of an ADC that is effective in the treatment of a particular disorder or condition will depend on the nature of the disorder or condition and may be determined by standard clinical techniques. In addition, in vitro or in vivo assays can optionally be employed to help identify optimal dosage ranges. The precise dose to be employed in the compositions will also depend on the route of administration, and the seriousness of the disease or disorder, and should be decided according to the judgment of the practitioner and each patient's circumstances.

The compositions comprise an effective amount of an ADC such that a suitable dosage will be obtained.

For intravenous administration, the composition may comprise from about 0.01 mg to about 100 mg of an ADC per kg of the patient's body weight. In one aspect, the composition can include from about 1 mg to about 100 mg of an ADC per kg of the patient's body weight. In another aspect, the amount administered will be in the range from about 0.1 mg/kg to about 25 mg/kg of body weight of the ADC.

Generally, the dosage of an ADC administered to a patient may typically be about 0.01 mg/kg to about 20 mg/kg of the patient's body weight. In one aspect, the dosage administered to a patient may be between about 0.01 mg/kg to about 10 mg/kg of the patient's body weight. In another aspect, the dosage administered to a patient may be between about 0.1 mg/kg and about 10 mg/kg of the patient's body weight. In yet another aspect, the dosage administered to a patient is between about 0.1 mg/kg and about 5 mg/kg of the patient's body weight. In yet another aspect the dosage administered is between about 0.1 mg/kg to about 3 mg/kg of the patient's body weight. In yet another aspect, the dosage administered is between about 1 mg/kg to about 3 mg/kg of the patient's body weight, or any other amount bounded by the ranges disclosed herein.

The ADC may be administered by any convenient route, for example by infusion or bolus injection. Administration may be systemic or local. Various delivery systems are known, e.g., encapsulation in liposomes, microparticles, microcapsules, capsules, etc., and may be used to administer an ADC. In certain embodiments, more than one ADC is administered to a patient.

In yet another embodiment, the ADC may be delivered in a controlled release system, such as but not limited to, a pump or various polymeric materials can be used. In yet another embodiment, a controlled-release system may be placed in proximity of the target of the ADC, thus requiring only a fraction of the systemic dose.

The term “carrier” refers to a diluent, adjuvant or excipient, with which an ADC is administered. Such pharmaceutical carriers may be liquids, such as water and oils, including those of petroleum, animal, vegetable or synthetic origin. The carriers may be saline, and the like. In addition, auxiliary, stabilizing and other agents may be used. In certain embodiments, when administered to a patient, the ADC and pharmaceutically acceptable carriers are sterile. Water is an exemplary carrier when the ADC are administered intravenously. Saline solutions, aqueous dextrose, and glycerol solutions may also be employed as liquid carriers, particularly for injectable solutions. The present compositions, if desired, may also contain minor amounts of wetting or emulsifying agents, or pH buffering agents.

The present compositions may take the form of solutions, pellets, powders, sustained-release formulations, or any other form suitable for use. Other examples of suitable pharmaceutical carriers are described in “Remington's Pharmaceutical Sciences” by E. W. Martin.

In certain embodiments, ADC are formulated in accordance with routine procedures as a pharmaceutical composition adapted for intravenous administration to animals, particularly human beings. Typically, the carriers or vehicles for intravenous administration are sterile isotonic aqueous buffer solutions. Where necessary, the compositions may also include a solubilizing agent. Compositions for intravenous administration may optionally comprise a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where an ADC is to be administered by infusion, it may be dispensed, for example, with an infusion bottle containing sterile pharmaceutical grade water or saline. Where the ADC is administered by injection, an ampoule of sterile water for injection or saline may be provided so that the ingredients can be mixed prior to administration.

The composition may include various materials that modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

### EXAMPLES

### Example 1

### Characterization of Broadly Neutralizing Anti-Gp41 Antibodies

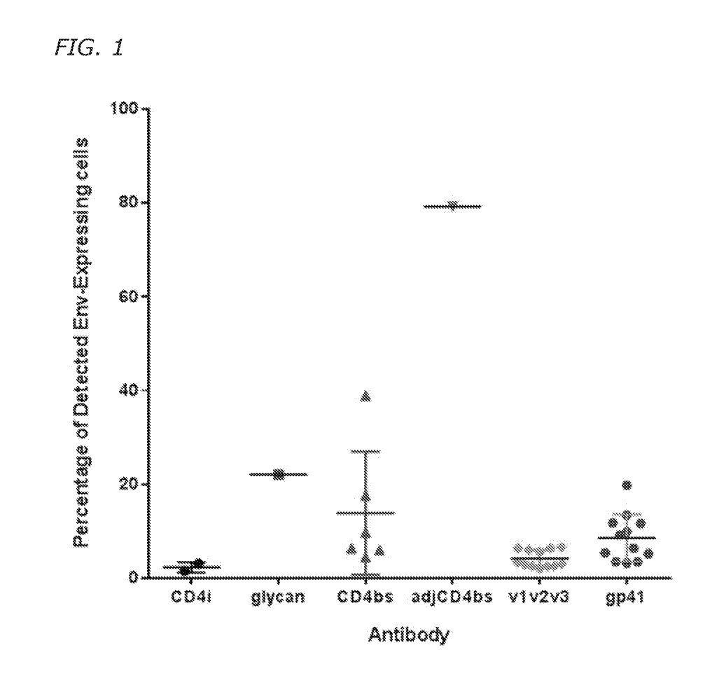

A large pool of mAbs available from the NIH AIDS Reagent Program were screened and it was determined that the NIH45-46 G54W antibody and its parent antibody NIH45-46 both bind to HIV-1 envelope glycoproteins gp120 and gp41. The protein and cell level evidence (FIG. 1-8) confirmed that NIH45-46 G54W binds to gp120 and gp41. The parental antibody, NIH45-46 also binds to gp41 with similar affinity, suggesting that the G54W mutation did not abrogate the binding to gp41. The epitope of this antibody overlaps the CD4 binding site (CD4bs) of gp120 and the trimer of gp41, however, its neutralization potency against different HIV-1 isolates was moderate. The dual recognition of gp120 and gp41 by a HIV-1 monoclonal antibody is rare since only limited regions of gp41 are accessible to antibodies on the native Env; other regions get exposed to the immune system only after gp120 shedding. In addition, cryptic gp41 epitopes are uncovered during viral fusion with the cell membrane. Multiple rounds of exposure to the immune system may drive B germ cells to gain additional recognition of gp120 and gp41. The binding sites of NIH45-46 G54W on gp41 was mapped using mAbs that target Clusters I to VI of gp41 ecto-domain. However, none of the well-known gp41 targeted antibodies could inhibit binding of NIH45-46 G54W to gp41. Thus, NIH45-46 G54W binds to an un-identified epitope on gp41.

The VRC01 and NIH45-46 antibodies are derived from donor VC10042 (Sather et al. J. Virol. 86:12676-85, 2012). The notable difference in protein sequence between VRC01 and NIH45-46 is four amino acid insertions in the CDRH3 of NIH45-46. These antibodies have significantly different neutralization potency, gp41 binding, and reactions to a panel of auto-antigens. The four amino acid differences between the two antibodies may contribute to the above-mentioned differences. A further modified version of NIH45-46, NIH45-46 G54W, includes a glycine to tryptophan mutation at amino acid 54.

NIH45-46 and NIH45-46 G54W bind to both the gp120 monomer and the gp140 trimer with a K_{D} of ˜10⁻¹° M and to gp41 at ˜10⁻⁹ M. On the cellular level, they also recognize the gp160 trimer and gp41 trimer alone. NIH45-46 and NIH45-46 G54W both also exhibit some marginal level of autoreactivity at high concentration when compared to VRC01. Fluorescence activated cell sorting (FACS) analysis was used to differentiate if the membrane recognition and additional gp41 binding may contribute to the enhanced recognition to the cell surface gp160 trimers. On the protein level, ELISA data and kinetic analysis by SPR confirmed that VRC01 and NIH45-46 G54W recognize the cleavable and non-cleavable gp140 trimer with similar potency. As a consequence, the recognition potency of VRC01 and NIH45-46 G54W in the context of cell membrane should be similar. However, the FACS analysis demonstrated that the recognition of cell-surface gp160 trimers by NIH45-46 G54W was significantly higher than VRC01, which indicate that lipid binding and/or additional gp41 binding may be involved in the enhanced recognition.

### Materials and Methods

Antibodies.

The human mAbs NIH45-46 G54W, 2F5, 4E10, 10E8, 2G12, b12, HJ16, VRC01, VRC03, 17b, and 19b were obtained from the NIH AIDS Research and Reference Reagent Program (ARRRP). MAbs D40, D50, D61, T3, T30 and m44 were produced in the inventors' laboratories. The hybridomas for MAbs D40, D50, D61, T3, and T30 were kindly provided by Dr. Patricia L. Earl. The following antibodies were purchased: HRP-conjugated rabbit anti-human IgG(Fc) antibody (Thermo Scientific, Rockford, Ill.), HRP-conjugated polyclonal anti-human IgG, F(ab′)₂ antibodies (Jackson ImmunoResearch, Westgrove, Pa.), FITC-conjugated goat anti-human IgG(Fc) antibody, FITC-conjugated goat anti-mouse IgG(Fc) antibody, PE-conjugated goat anti-mouse IgG(Fc) antibody, PE-conjugated goat anti-human IgG(Fc) antibody (Thermo Scientific) and HRP-conjugated streptavidin (Zymed Laboratories Inc., San Francisco, Calif.).

The light and heavy chain amino acid sequences of NIH45-46 G54W are as follows: (variable regions are underlined)

Light chain
(SEQ ID NO: 1)
EIVLTQSPAT LSLSPGETAI ISCRTSQSGS LAWYQQRPGQ
APRLVIYSGS TRAAGIPDRF SGSRWGADYN LSISNLESGD
FGVYYCQQYE FFGQGTKVQV DIKRTVAAPS VFIFPPSDEQ
LKSGTASVVC LLNNFYPREA KVQWKVDNAL QSGNSQESVT
EQDSKDSTYS LSSTLTLSKA DYEKHKVYAC EVTHQGLSSP
VTKSFNRG
Heavy chain
(SEQ ID NO: 2)
QVRLSQSGGQ MKKPGESMRL SCRASGYEFL NCPINWIRLA
PGRRPEWMGW LKPRWGAVNY ARKFQGRVTM TRDVYSDTAF
LELRSLTSDD TAVYFCTRGK YCTARDYYNW DFEHWGRGAP
VTVSSASTKG PSVFPLAPSS KSTSGGTAAL GCLVKDYFPE
PVTVSWNSGA LTSGVHTFPA VLQSSGLYSL SSVVTVPSSS
LGTQTYICNV NHKPSNTKVD KKVEPKSCDK THTCPPCPAP
ELLGGPSVFL FPPKPKDTLM ISRTPEVTCV VVDVSHEDPE
VKFNWYVDGV EVHNAKTKPR EEQYNSTYRV VSVLTVLHQD
WLNGKEYKCK VSNKALPAPI EKTISKAKGQ PREPQVYTLP
PSRDELTKNQ VSLTCLVKGF YPSDIAVEWE SNGQPENNYK
TTPPVLDSDG SFFLYSKLTV DKSRWQQGNV FSCSVMHEAL
HNHYTQKSLS LSPGK

Proteins.

Recombinant HIV-1 gp120IIIB, gp120BaL, gp41Yu2, gp41BaL, and four-domain sCD4 were purchased from ImmunoDX, LLC. Recombinant gp41HXB2 (ecto-domain aa546 to 682) and biotin-conjugated HIV-1 gp41HXB2 were purchased from Aviva System Biology Corp. Recombinant HIV-1 gp41MN, SF162 gp140 trimer, and two-domain sCD4 were obtained from the NIH AIDS Reagent Program (ARP). The full-length Yu2 gp120 protein was kindly provided by Dr. Peter D. Kwong of the Vaccine Research Center at the NIH. BG505 SOSIP.664 gp140 trimer was kindly provided by Dr. John P Moore of the Weill Cornell Medical College.

Cells, Viruses and Plasmids.

293T cells, BHK-21 cells and CV-1 cells were purchased from ATCC. vP1340, a recombinant vaccinia virus expressing the gp41 portion of HIV-1_{MN} Env was provided by Virogenetics Corp. ACH-2, A3.01 and vTF7-3, a recombinant vaccinia virus expressing bacteriophage T7 RNA polymerase, were obtained from the NIH ARP. The cleavage-competent JRFL gp160 plasmid was kindly provided by Dr. Joseph Sodroski of Harvard Medical School. The plasmid encoding m44 was kindly provided by Dr. Dimiter Dimitrov of NIH/Frederick. The plasmid vectors expressing heavy chain of VRC01 and light chain of VRC01 were obtained from the NIH ARP. The plasmid vectors expressing heavy chain and light chain of NIH45-46 or NIH45-46 G54W were synthesized by DNA 2.0 (Menlo Park, Calif.).

Production of Purified Recombinant MAbs.

The NIH45-46 and NIH45-46 G54W Ig V_{H} and V_{K} genes were synthesized by DNA 2.0 and were subcloned into human b12 Igγ and Igκ expression back-bone vectors. NIH45-46 G54H Ig V_{H} was generated by directed mutation. Clones with the correct size inserts were sequenced to confirm identity with the original gene fragments. For production of purified antibodies of m44, VRC01, NIH45-46, and NIH45-46 G54W by batch transient transfections, 10-20 T-175 flasks of 293T cells grown at 70-80% confluency in serum-free media were co-transfected with plasmids expressing HIV-1 specific Ig heavy- and light-chain genes using FUGENE® HD (Roche, Nutley, N.J.). Recombinant antibodies were purified using Protein A/G affinity chromatography (Thermo Scientific) and size exclusion chromatography.

Hybridomas were grown to high density in Iscove's minimal essential medium containing 10% FCS. Cells were maintained for several days with daily additions of medium. IgG was purified by protein A/G affinity chromatography and size exclusion chromatography and concentrated in CENTRICON® microconcentrators (Merck KGa, Darmstadt, Germany). The protein concentration was determined after the antibody buffer was changed. IgG preparations were biotinylated with NHS-SS Biotin (Thermo Scientific) according to the manufacturer's specifications. Briefly, the IgG1 antibody (1 to 2 mg/ml) was equilibrated in 50 mM sodium bicarbonate (pH 8.5). Freshly prepared biotin was added at 20-fold molar excess. After 45 min at room temperature, the reaction was quenched by addition of 4 to 10 volumes of 10 mM sodium phosphate-150 mM NaCl (pH 7.2). IgG was then concentrated in CENTRICON® microconcentrators, and antibody concentration was determined from the A₂₈₀.

Binding Assays with Recombinant Env Glycoproteins or Peptides.

The antibody binding to gp120, gp41 or gp140-trimer proteins was tested by ELISA. Briefly, high-binding 96-well ELISA plates were coated overnight at 4° C. with 100 ng/well of gp120, gp41, or gp140-trimer in PBS. After washings, plates were blocked 2 h with 2% BSA, 1 μM EDTA, 0.05% TWEEN®-PBS (blocking buffer). Pre-coated plates were incubated 2 h with IgG antibodies diluted at 8 μg/ml, 4 μg/ml, and six consecutive 1:4 dilutions in PBS. After washings, the plates were developed by incubation for 1 h with rabbit HRP-conjugated anti-human IgG (Thermo Scientific) (at 0.8 μg/ml in blocking buffer) and by adding 100 μl of HRP chromogenic substrate (TMB solution, Sigma) after washing steps. Optical densities were measured at 405 nm (OD_{405 nm}) or at 450 nm (OD_{450 nm}) using an ELISA microplate reader (Tecan, Morrisville, N.C.). Background values given by incubation of PBS alone in coated wells were subtracted. All ELISA experiments were performed at least in duplicates.

To assay the antibody binding to gp41 MPER (membrane-proximal external region) peptides, the anti-Env IgG antibodies were tested using a peptide-ELISA method. Briefly, ELISA plates were coated with 50 μl of the peptide recognized by 2F5 (SQNQQEKNEQELLALDKWAS, SEQ ID NO:3) underlining refers to the minimal epitope) or 4E10 (LWNWFDITKWLWYIKIFIMI, SEQ ID NO:4) (both purchased from CPC Scientific, Inc, Sunnyvale, Calif.) at 5 μg/ml in PBS and incubated overnight at room temperature. After plates were washed three times with PBS-0.1% TWEEN® 20 (PBST), wells were blocked with PBS-1% TWEEN® 20-5% sucrose-3% milk powder for 1 h at room temperature. Serial dilutions of human IgG (starting at 8 μg/ml in PBST-1% bovine serum albumin [BSA]) were added, and samples were incubated for 1 h at room temperature and visualized with peroxidase-conjugated affinity-purified rabbit anti-human IgG (Thermo Scientific). Controls were VRC01, 2F5, and 4E10 (each of which was included in every experiment).

### Results

Identification of mAb that Recognizes Cell Surface Envelope Glycoprotein with High Affinity.

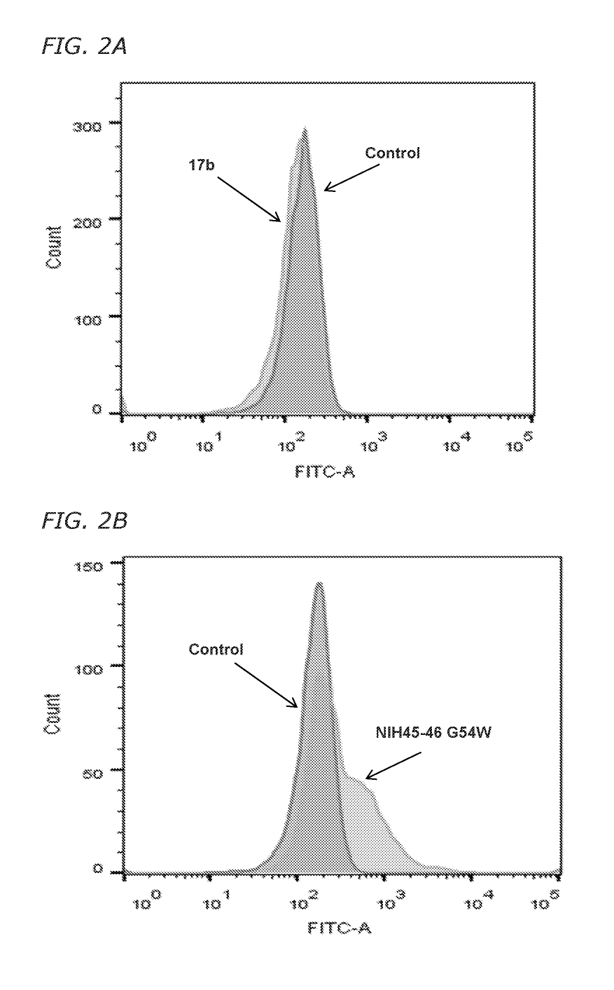

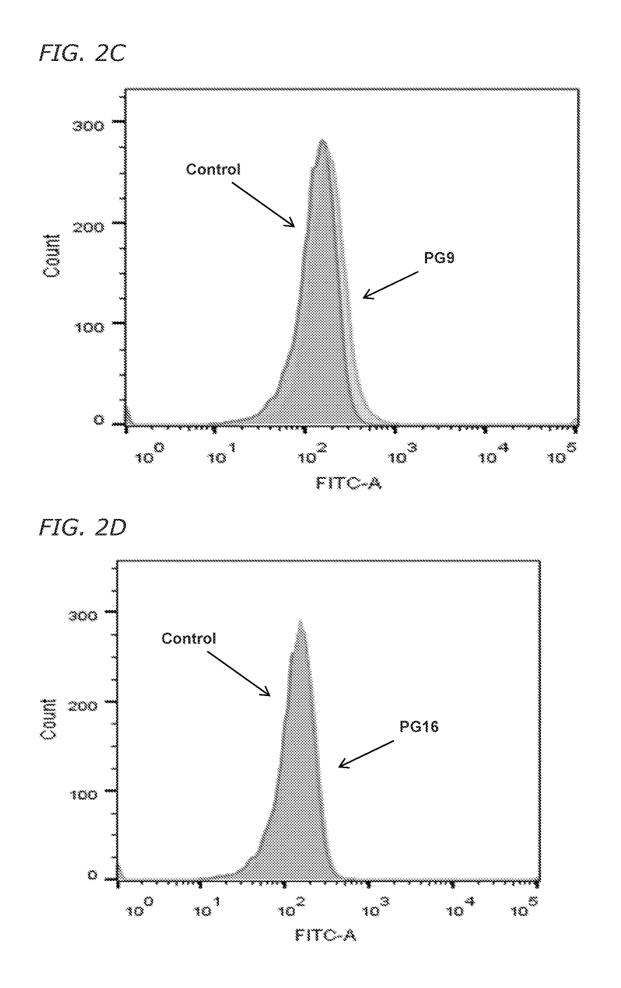

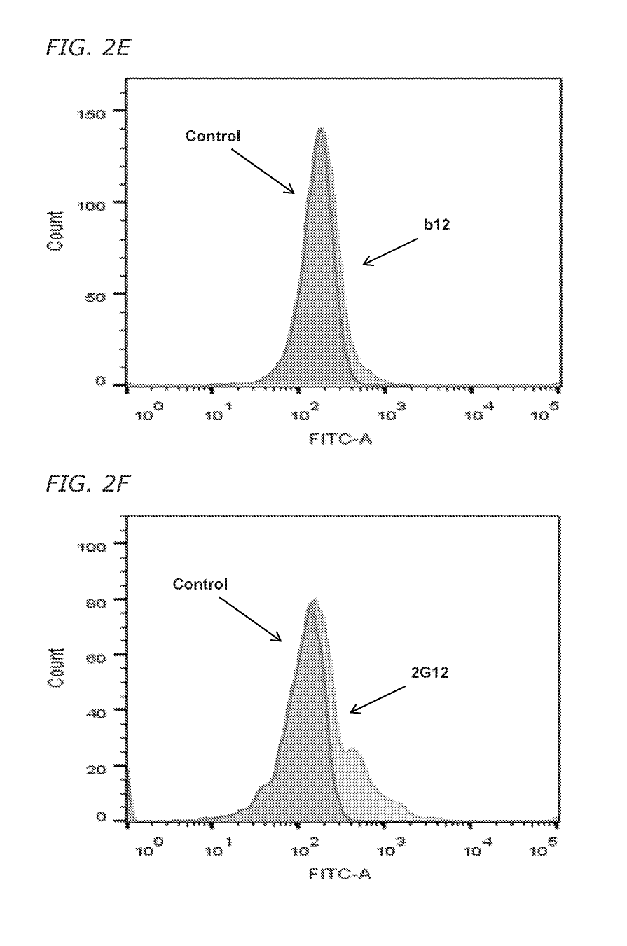

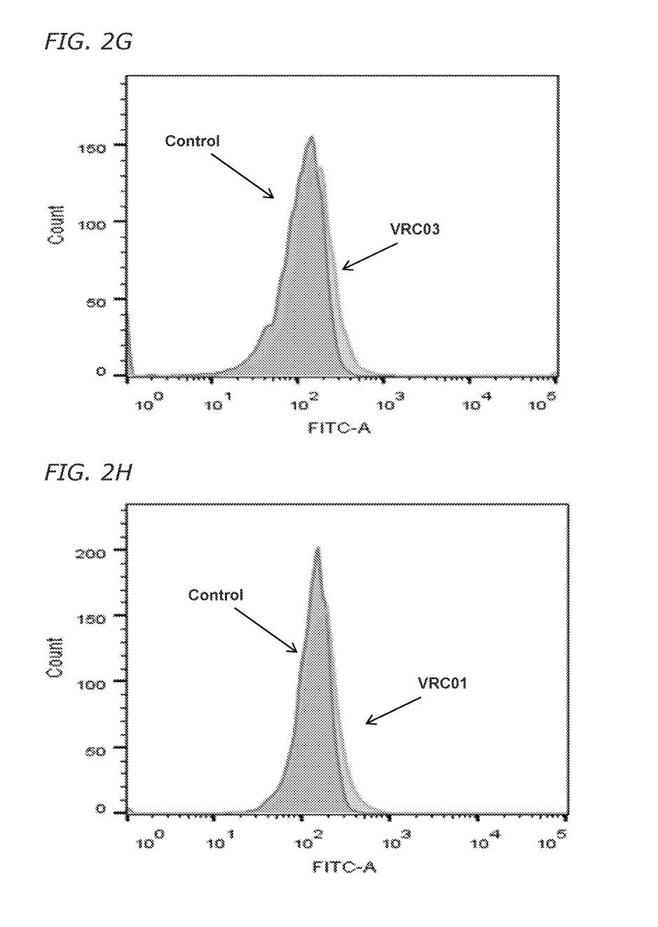

Thirty-four mAbs including bNAbs available from the NIH AIDS Reagent Program (ARP), specifically targeted to the envelope glycoprotein of HIV-1, were screened to identify mAbs with the high recognition of cell surface Env. Among those screened were two CD4-inducing (CD4i) mAbs, six recognizing the CD4-binding site (CD4bs), twelve recognizing the variable loop (V1, V2 and/or V3), twelve against gp41, one recognizing glycan, and one recognizing a site adjacent to CD4bs (adjCD4bs). 293T cells were transfected with Yu2gp160 to express cell surface Env and the mAb recognition of Env was analyzed by FACS (Table 1, FIG. 1, FIG. 2A-H). The results in FIG. 1 indicate that the adjCD4bs mAb (A32) showed the highest recognition of the surface-expressing Env on the cell surface (79.20%), followed by the CD4bs, gp41 and V1, V2, and/or V3 loop targeted mAbs. The glycan targeted antibody (2G12) showed somewhat better recognition than gp41 and V1, V2, and/or V3 loop targeted mAbs. The mAbs from the V1, V2, and/or V3 targeted group including the two most potent bNAbs; PG9 and PG16, showed the poorest recognition of the cell surface Env. Monoclonal antibodies from the CD4i, glycan and adjCD4bs were not included in the statistical analysis since the sample number was too small.

A32, an antibody directed to the binding site adjacent to CD4 (adjCD4-bs) and NIH45-46 G54W, an antibody directed to CD4bs, have the highest avidity to the cell surface Env proteins. This indicates that these two antibodies can sample many conformers of Env proteins with higher avidity. However, further studies showed that A32 can bind to un-infected cells and may cause autoreactivity (Table 1); therefore, this mAb was not considered for further study. Among all the highest cell surface Env recognizing mAbs, NIH 45-46 G54W showed the least autoreactivity (Table 1), thus this bNAb was further characterized biochemically and functionally in order to gain basic information to target the Env on the cell surface rather than on the viral surface.

TABLE 1
Screening of monoclonal antibodies obtained from
the NIH AIDS Reagent Program.

| | | Percentage | |
|---|---|---|---|
| | | of | |
| | | recognition | Percentage |
| | | of | auto- |
| Antibody Name | Target | HIV-1 Env | reactivity |
| HIV-1 gp120 Monoclonal | CD4i | 1.50% | 0.10% |
| Antibody (17b) | | | |
| HIV-1 gp120 Monoclonal | CD4i | 3.10% | |
| Antibody (48d) | | | |
| HIV-1 gp120 Monoclonal | glycan | 22.80% | 0.20% |
| (2G12) | | | |
| HIV-1 gp120 Monoclonal | CD4bs | 4.50% | |
| Antibody (F105) | | | |
| HIV-1 gp120 Monoclonal | CD4bs | 9.80% | 0.30% |
| Antibody (IgG1 b12) | | | |
| HIV-1 gp120 MAb (VRC03) | CD4bs | 6.40% | |
| HIV-1 gp120 MAb (VRC01) | CD4bs | 6.00% | |
| HIV-1 gp120 MAb (HJ16) | CD4bs | 17.60% | 0.30% |
| HIV-1 MAb NIH45-46 | CD4bs | 38.90% | 0.80% |
| G54W IgG | | | |
| HIV-1 gp120 MAb (A32) | adjCD4bs | 79.20% | 1.50% |
| Monoclonal Antibodies | v3 | 2.10% | |
| to HIV-1 V3 (268) | | | |
| HIV-1 gp120 Monoclonal | v3 | 2.50% | |
| Antibody (F425 B4a1) | | | |
| HIV-1 gp120 Monoclonal | v3 | 6.40% | |
| Antibody (F425 B4e8) | | | |
| HIV-1 gp120 MAb (39F) | v3 | 2.60% | |
| HIV 1 V3 Monoclonal | v3 | 2.90% | |
| Antibody (2191) | | | |
| HIV 1 V3 Monoclonal | v3 | 3.10% | |
| Antibody (2219) | | | |
| HIV 1 V3 Monoclonal | v3 | 6.60% | |
| Antibody (2442) | | | |
| HIV 1 V3 Monoclonal | v3 | 2.60% | |
| Antibody (3869) | | | |
| HIV 1 V3 Monoclonal | v3 | 3.40% | |
| Antibody (3074) | | | |
| HIV-1 V2V3 Monoclonal | v2v3 | 5.60% | |
| Antibody (2909) | | | |
| HIV-1 MAb PG9 | v1v2 | 6.00% | |
| HIV-1 MAb PG16 | v1v2 | 1.50% | |
| HIV-1 gp41 Monoclonal | gp41 | 9.90% | 0.70% |
| Antibody (2F5) | | | |
| HIV-1 gp41 Monoclonal | gp41 | 3.50% | |
| (98-6) | | | |
| HIV-1 gp41 Monoclonal | gp41 | 6.40% | |
| Antibody (5F3) | | | |
| HIV-1 gp41 Monoclonal | gp41 | 3.50% | |
| Antibody (F240) | | | |
| HIV-1 gp41 Monoclonal | gp41 | 3.20% | |
| (126-7) | | | |
| HIV-1 gp41 Monoclonal | gp41 | 13.40% | 1.40% |
| Antibody (4E10) | | | |
| HIV-1 gp41 MAb | gp41 | 5.20% | |
| (IgG1 Z13e1) | | | |
| HIV 1 gp41 Monoclonal | gp41 | 11.80% | 0.60% |
| Antibody (167-7, 67-D IV) | | | |
| HIV-1 gp41 Monoclonal | gp41 | 4.50% | |
| Antibody (50-69) | | | |
| HIV-1 anti-gp41 mAb (10E8) | gp41 | 11.70% | 0.80% |
| HIV-1 anti-gp41 mAb (7H6) | gp41 | 19.80% | 0.60% |

NIH45-46 G54W, NIH45-45, and VRC01 Bind to Both Cleaved gp140 Trimer (BG505 SOSIP.646) and Uncleaved SF162 gp140 Trimer.

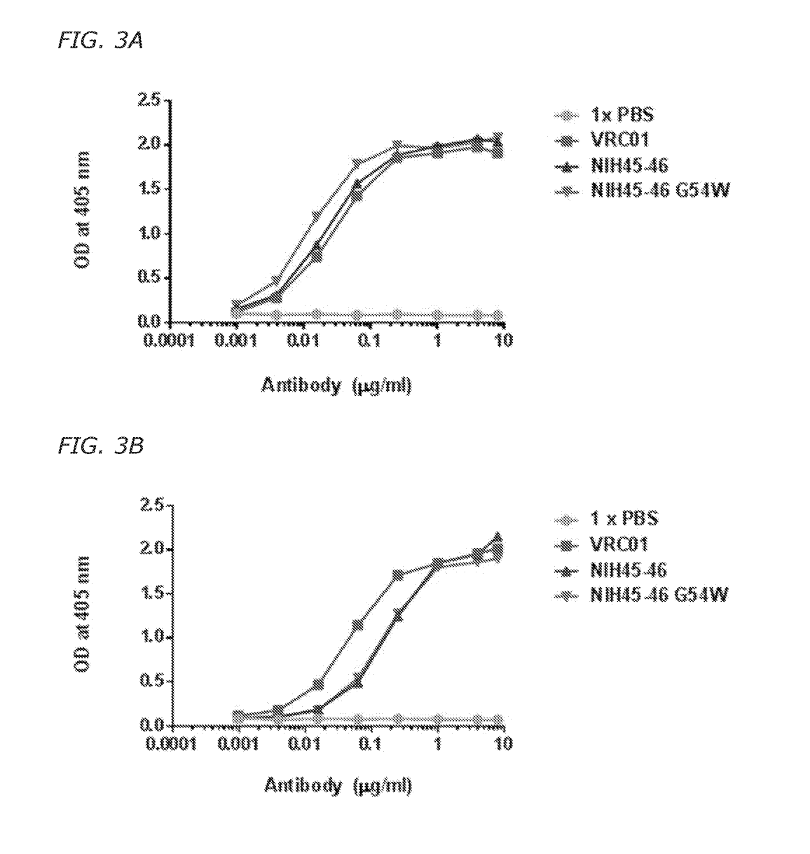

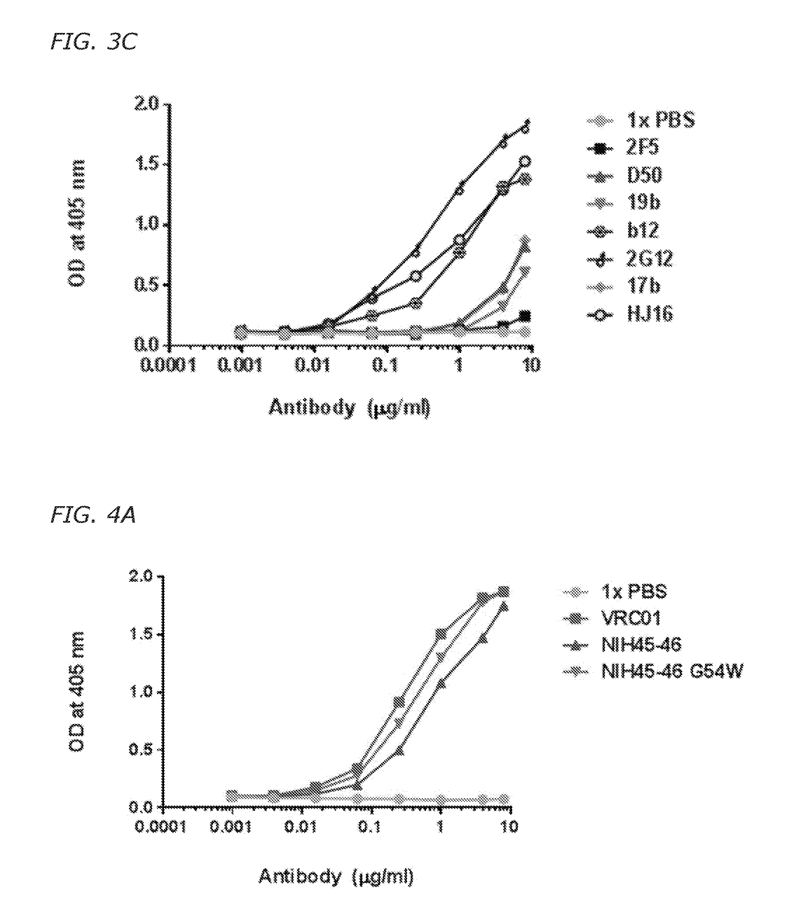

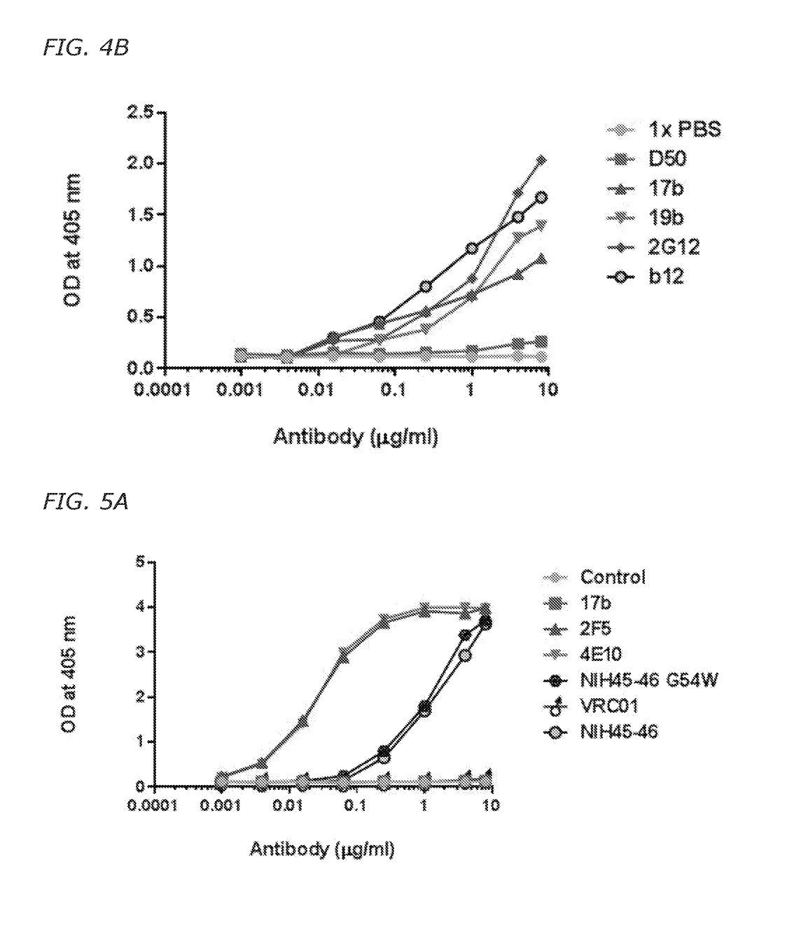

To gain insight on how NIH45-45 G54W can achieve maximal sampling of the cell surface Env proteins, the binding affinity of NIH45-46 G54W to two trimeric Env proteins, cleavable and non-cleavable Env trimer, was evaluated. BG505 SOSIP.646 is one of the recent cleavable and stable versions of gp140 trimer. It has been reported that the uncleaved gp160 trimers are also present on the cell surface, therefore, a non-cleavable version of gp140 trimer (SF162) was also included to probe bNAbs. Highly neutralizing bNAbs (VRC01 and NIH45-46), moderately neutralizing mAbs, (b12, HJ16, 2G12), and non-neutralizing mAbs (17b, 19b and D50) were used as controls. NIH45-46 G54W, NIH45-46, and VRC01 showed high affinity binding to both BG505 SOSIP.664 trimer (FIG. 3A) and SF162 trimer (FIG. 3B). However, these bNAbs showed somewhat higher affinity to BG505 SOSIP.664 trimer. The control antibodies, HJ16, 2G12 and b12 bound to BG505 SOSIP.646 trimer at a much lower affinity as expected, whereas mAbs 17b, 19b, 2F5, and D50 bound to BG505 SOSIP.646 trimer at a significantly lower affinity (FIG. 3C). The binding of bNAbs and the control antibodies was comparted to monomeric full-length gp120 in order to gain further insight on the preference of binding of these antibodies to gp140 trimer and gp120 monomer. As expected and shown in FIG. 4A, NIH45-46 G54W, NIH45-46, and VRC01 bound to full-length gp120 monomer with similar affinity; however, they bound to gp140 trimer with higher affinity than monomer. The control antibodies, 2G12 and b12 prefer to bind to the full-length gp120 monomer (FIG. 4B) over gp140 trimer (FIG. 3B). The moderately neutralizing antibody 19b and weakly neutralizing antibody 17b exhibited tight binding to the full-length gp120 monomer. D50, a non-neutralizing gp41-directed antibody exhibited no binding, as expected, to the full-length gp120 monomer.

NIH45-46 and NIH45-46G54W Bound to Gp41 while VRC01 does not.

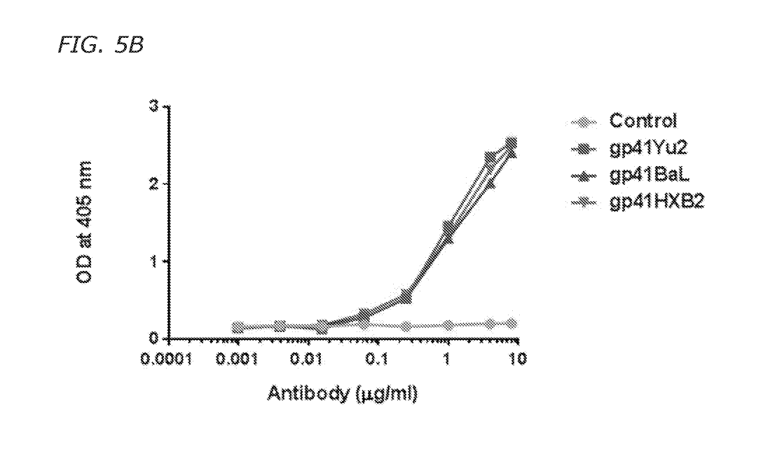

In the antibody screening program, ELISA was used to estimate the binding of all antibodies described supra to gp120 and/or gp41 on the molecular level. NIH45-46 G54W was initially chosen as a negative control to estimate 10E8 binding to gp41. Unexpectedly, NIH45-46 G54W and NIH45-46 bound to gp41 from HIV-1 MN when compared to the binding by 2F5, 4E10, and 10E8, all gp41 targeted mAbs, while VRC01, one of the earlier variants of NIH45-46, showed no binding to gp41 MN (FIG. 5A). 17b, a CD4i antibody which recognizes the CCR5 coreceptor binding site in gp120, showed no binding to gp41MN. The ELISA experiment was further extended to gp41 from HIV-1 Yu2, HIV-1 BaL, and HIV-1 HXB2 ecto-domain. As FIG. 5B shows, NIH45-46 G54W retains comparable binding affinity to gp41 from different strains of HIV-1, which suggests that NIH45-46 G54W binds to the conserved epitope on gp41. Combined with the results presented earlier, the data indicate that NIH45-46 G54W and NIH45-46 bind to both gp120 and gp41.

Mapping of the Binding Site of NIH45-46 G54W on Gp41.

The binding site of NIH45-46 G54W on gp41 ecto-domain was mapped by using two sets of ELISAs. To determine if NIH45-46 G54W recognizes the epitopes of the two well-characterized gp41 targeted mAbs 2F5 and 4E10, ELISA was performed to detect binding between NIH45-46 G54W and peptides bearing the 2F5 or 4E10 epitopes. Monoclonal 4E10 and 2F5 were included as controls. NIH45-46 G54W did not bind to the peptide bearing the 2F5 or 4E10 epitopes even at very high concentrations (8 μg/ml).

To further characterize the epitopes recognized by NIH45-46 G54W antibody, competition ELISAs were carried out with well-characterized anti-gp41 antibodies. The following anti-gp41 antibodies were used as standards: D61, which recognizes an amino-terminal determinant corresponding to the immunodominant region, and spans amino acids 597 to 613 (cluster I; LAI strain); D40 and D50, which bind to a region that includes amino acids 642 to 665 located at the amino terminus of the MPER (cluster II); 4E10 and 2F5, which bind to separate but adjacent peptides in the MPER that span amino acids 662 to 678 (cluster III); T3, which binds a conformational epitope neighboring the MPER and spans amino acids 641 to 683 (cluster IV); and T30, which binds to a highly glycosylated region and mapped to amino acid 616, which is a critical glycosylation site (cluster VI). None of the listed and well-characterized gp41 antibodies inhibited the binding of NIH45-46 G54W to gp41. The data indicate that the gp41 binding site of NIH45-46 G54W is located outside the cluster I to VI regions.

Binding Affinity of NIH45-46 and NIH45-46 G54W.

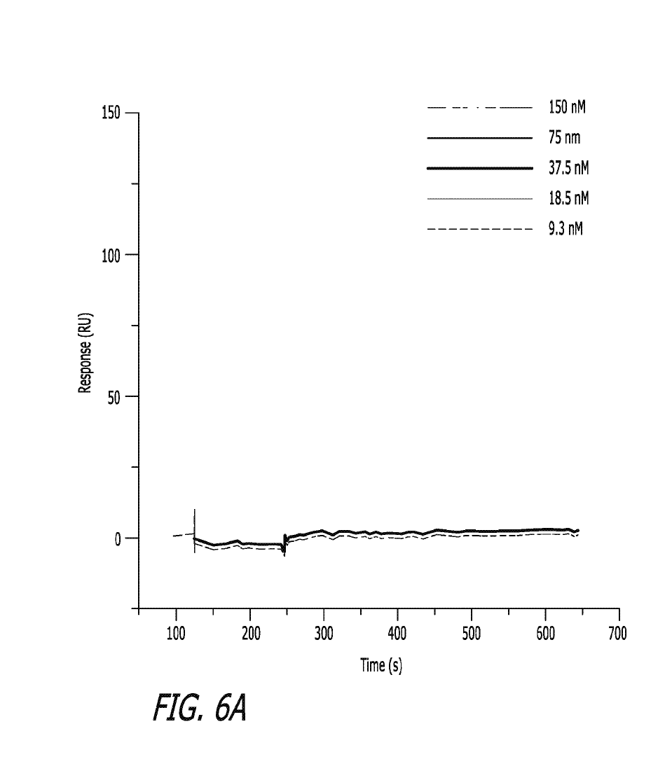

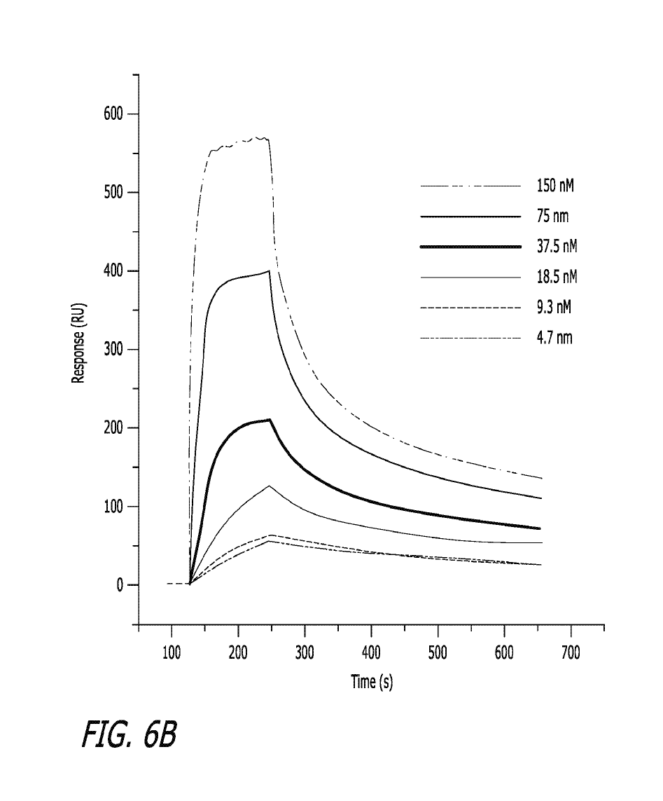

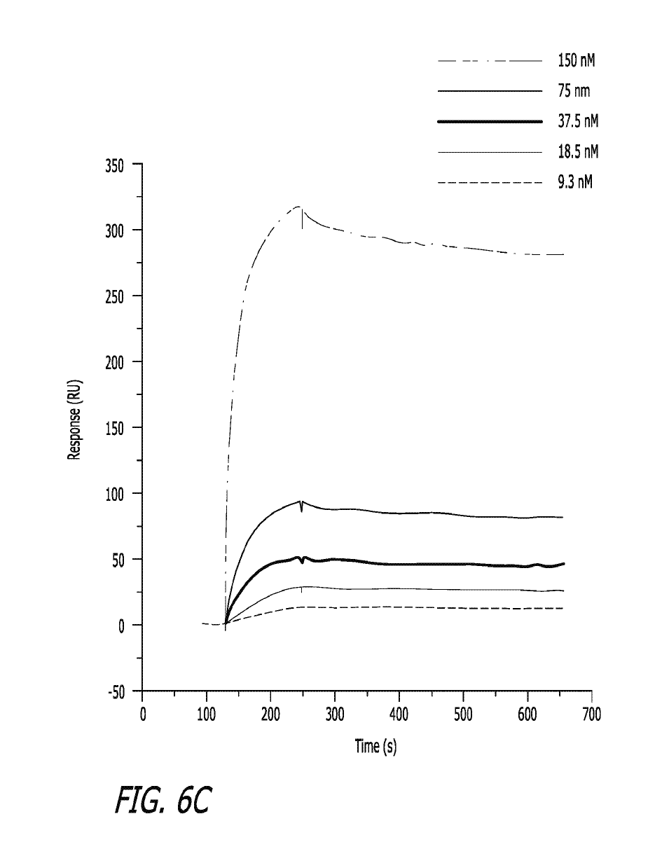

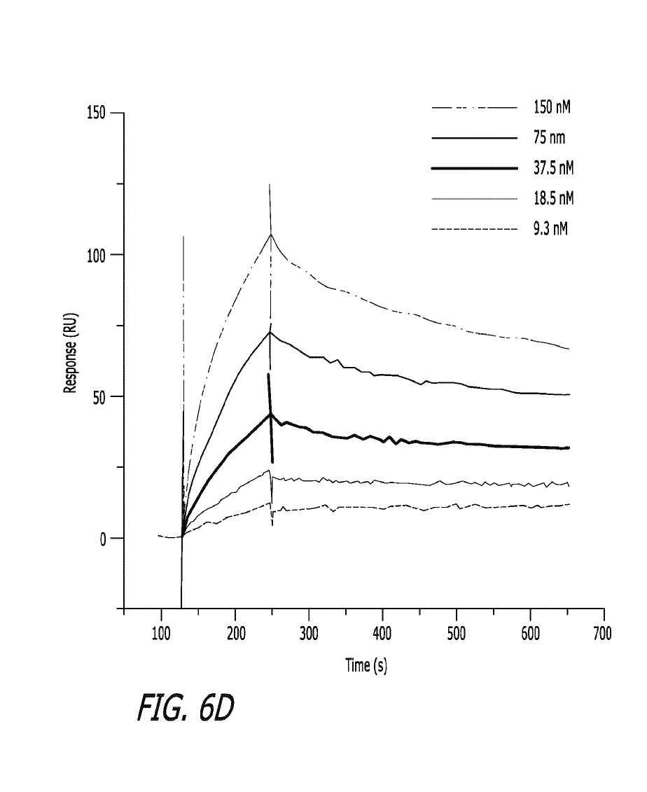

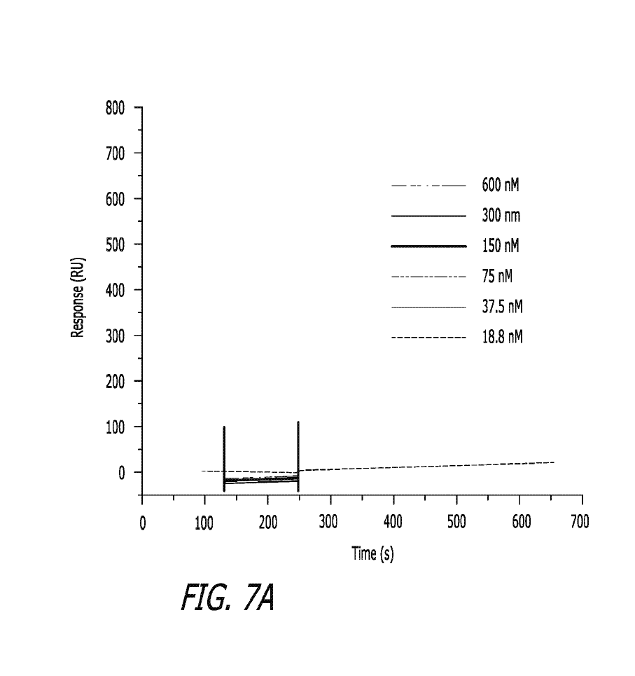

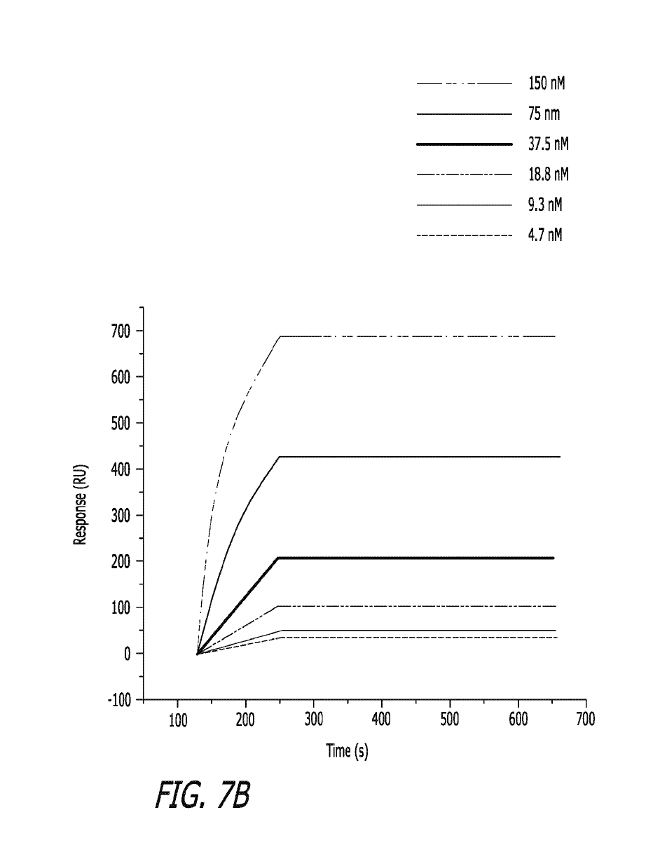

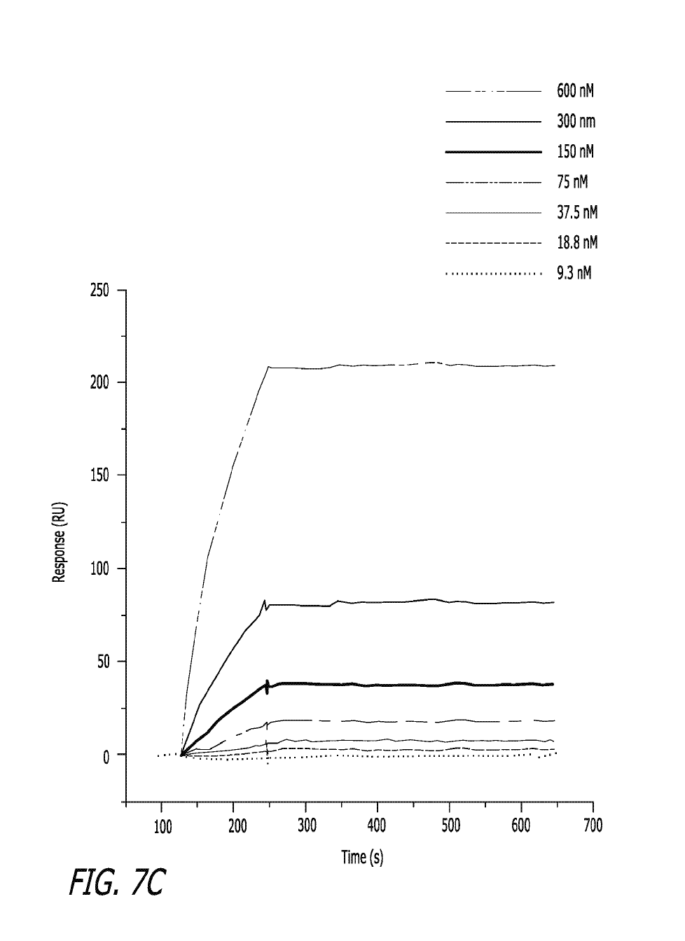

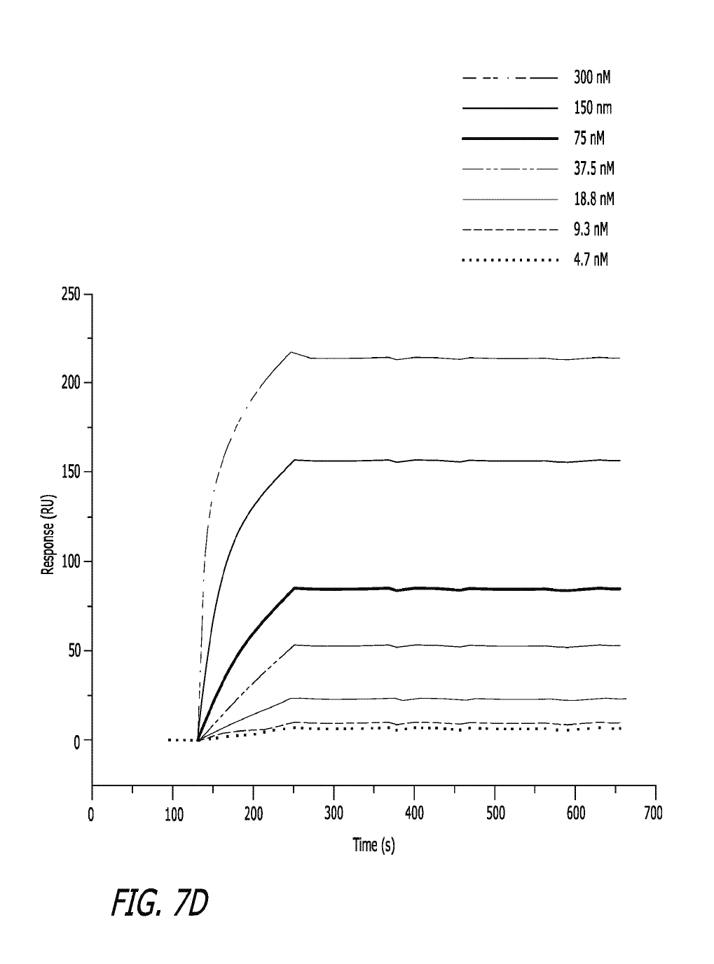

Surface plasmon resonance (SPR) analysis by BIAcore (GE Healthcare, Little Chalfont, UK) was used to quantify and validate the ELISA binding data reported above. The SPR data indicate that NIH45-46 G54W binds tightly to gp41 from HIV-1 strains MN, IIIB, Yu2, and HXB2 (ectodomain) with K_{D} ranging from 6.5 nM to 7.8 nM. Representative data is shown in Table 2, and FIG. 6A-D, which indicate faster on-rate and faster off-rate of the binding of NIH45-46 G54W unlike the positive control mAbs 10E8 and 4E10, two well-known gp41-targeted human mAbs (FIG. 6C-D). The binding affinity (K_{D}) of NIH45-46 G54W to gp41 is ˜4-5-fold lower than the binding of mAbs 10E8 and 4E10. Again, mAb 17b, a known gp120 CD4i binding human antibody was used as negative control and it showed no binding to gp41 in the BIAcore experiments (Table 1 and FIG. 6A). The binding of NIH45-46 G54W to full-length gp120 and cleavable version of gp140 trimer (BG505 SOSIP.646) was also quantified by SPR. The NIH45-46 G54W demonstrated the tightest binding to gp140 trimer with K_{D} ranging from 0.23 nM to 0.29 nM (FIG. 7AB). The binding of NIH45-46 G54W to full-length gp120 compared to the control mAb 17b (FIG. 7C) was much tighter (K_{D}=0.11 to 0.17 nM) (FIG. 7D), which is comparable to the binding to cleavable version of gp140 trimer. Binding affinities of NIH45-46 (FIG. 7D) and 10E8 (FIG. 7A) are also shown. The kinetic analysis on the binding affinity of NIH45-46 G54W to gp120 monomer and gp140 trimer supported ELISA results and confirmed that NIH45-46 G54W bind to both gp120 monomer and cleavable version of gp140 trimer with high affinity.

TABLE 2
Kinetic rates (kₒₙ and k_{off}) and binding affinity between Env proteins and antibodies

| Ligand | Antibody | kₒₙ (M⁻¹, s⁻¹) | k_{off} (s⁻¹) | KD (M) | Rmax(RU) |
|---|---|---|---|---|---|
| gp140-cleavable | 10E8 | — | — | no binding | |
| trimer | NIH45-46 | 2.01 × 10⁴ | 5.37 × 10⁻⁶ | 2.67 × 10⁻¹⁰ | 1080 |
| | G54W | | | | |
| gp120 | 17b | 2.64 × 10³ | 2.59 × 10⁻⁶ | 9.82 × 10⁻¹⁶ | 230 |
| | NH45-46 | | | | |
| | G54W | 4.48 × 10⁴ | 4.94 × 10⁻⁶ | 1.10 × 10⁻¹⁶ | 259 |
| gp41 | 17b | — | — | no binding | |
| | 4E10 | 1.98 × 10³ | 2.14 × 10⁻⁵ | 1.08 × 10⁻⁸ | 377 |
| | 10E8 | 5.80 × 10⁻⁴ | 9.01 × 10⁻⁴ | 1.55 × 10⁻⁸ | 118 |
| | NIH45-46 | 6.83 × 10⁵ | 4.61 × 10⁻³ | 6.75 × 10⁻⁹ | 591 |
| | G54W | | | | |

Reactivity of NIH45-46 G54W with Intact Gp160 Protein Trimer and Gp41 Protein Trimer Alone on the Cell Surface.

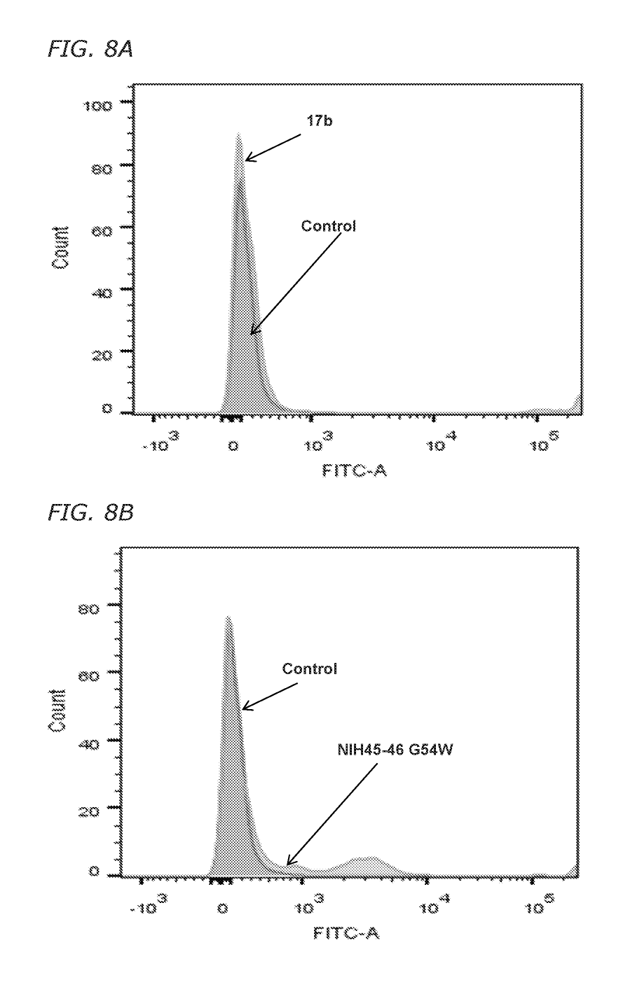

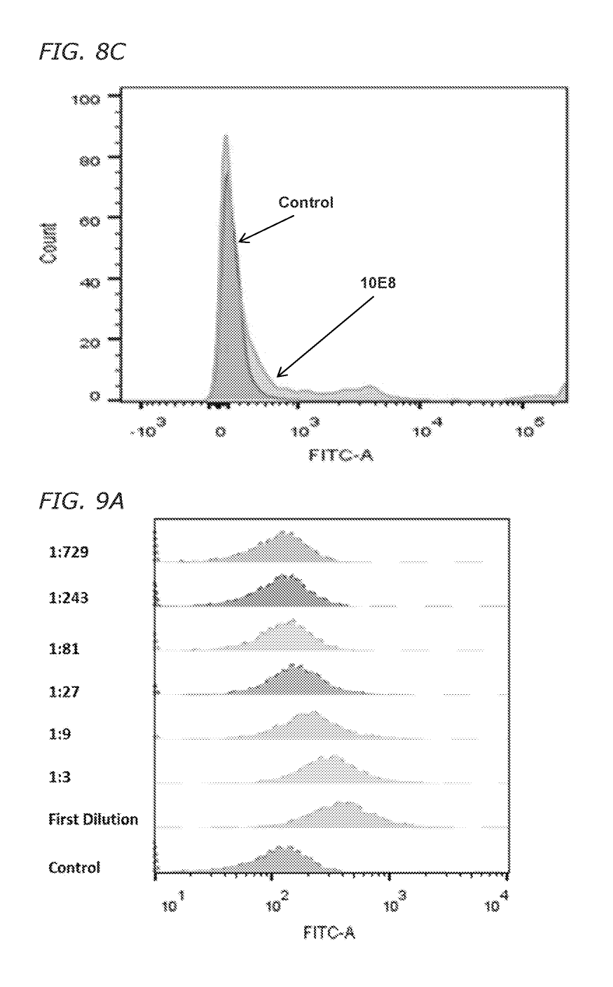

Two sets of FACS analyses were used to test the binding of NIH45-46 G54W and VRC01 to either the full-length Env trimer or gp41 trimer alone on the cell surface. On the protein level, NIH45-46 G54W and VRC01 bind with high affinity to both cleavable and non-cleavable version of gp140 trimer. The accumulated evidence indicates that the uncleaved gp160 trimers are present on the cell-surface. NIH45-46 G54W demonstrated significantly higher recognition to the cell surface HIV-1 Yu2 gp160 than VRC01 (38.9% vs 6.0%) (FIG. 2B vs. FIG. 2H). Monoclonal antibodies 17b and 19b were used as control and exhibited significantly lower recognition to the cell surface gp160 Yu2 trimer which is consistent with their binding avidity to gp140 trimer. In the second set of FACS analyses, gp41 was used as the only HIV viral protein expressed on the CV-1 cell surface. As shown in FIG. 8A-C, NIH45-46 G54W demonstrated a comparable recognition of the cell surface functional gp41 to the gp41 targeted mAb 10E8. The control mAb 17b showed no recognition to the cell surface functional gp41. mAbs 17b, 10E8, and NIH45-46 G54W did not show any recognition to the T7-polymerase-expressing CV-1 cells infected with the recombinant vaccinia virus, vTF7-3, which indicated the recognition is specific.

Binding of NIH45-46 G54W to Reactivated ACH-2 Latent Cells.

NIH45-46 G54W binds with high affinity to full length gp120 and both cleavable and non-cleavable gp140 trimer. NIH45-46 G54W was used to probe the recognition efficiency of cell surface Env expressed on the ACH-2 cells after 3 days activation by TNF-α. NIH45-46 G54W showed 40-60% efficiency in recognizing the Env expressed on the activated ACH-2 cells, which conform to the data obtained with Env-expressed 293T cells. Furthermore, the dosage required to achieve the half-maximal Env-recognition was determined as shown in FIGS. 9A and 9B (EC₅₀ of 5.2 μg/ml).

Uptake of NIH45-46 G54W in Reactivated ACH-2 Latent Cells.

HIV and SIV Env proteins either bud from the host cell membrane or become an important component on the virion surface after the virus particle is released or rapidly internalize to the endosome by endocytosis. The internalization of Env results into low number of Env incorporated in the virus particles, a strategy adopted by viruses to avoid immune recognition and clearance. To investigate if NIH45-46 G54W can take advantage of the internalization pathway after binding to the Env of the cell surface and be taken up by cells, FITC-conjugated NIH45-46 G54 was incubated with the activated ACH-2 latent cells at different intervals and analyzed by flow cytometry analysis after fixing in 1% paraformalin. To verify whether NIH45-46 G54W is taken up by Env-expressing cells, 8 μg/ml of FITC-conjugated NIH45-46 G54W was incubated with 1×10⁶ of activated ACH-2 cells for 0, 15 min, 1 h, 2 h or 4 h. Following 3 washes with 1×PBS, the cells were fixed with 1% paraformaldehyde and studied by flow cytometry.

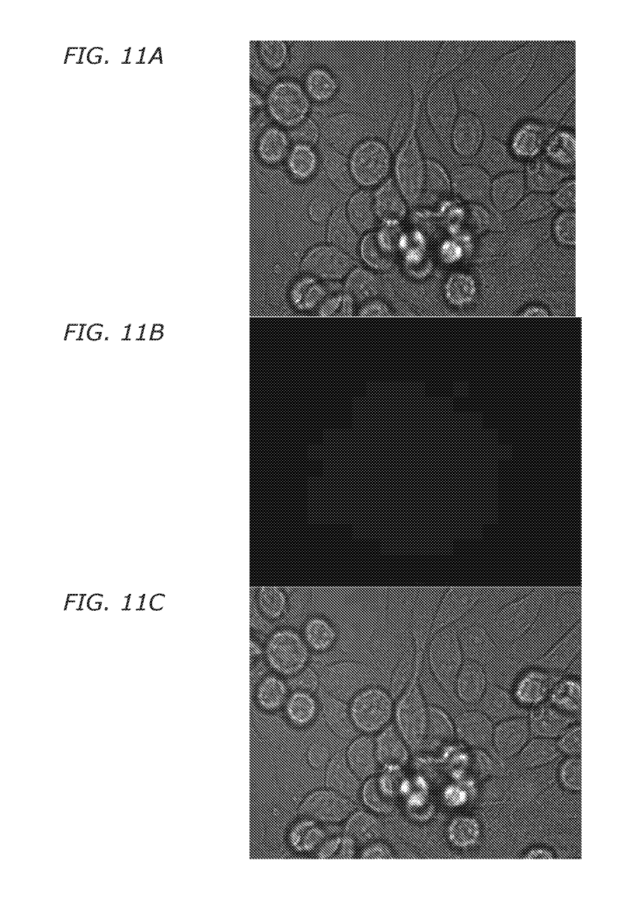

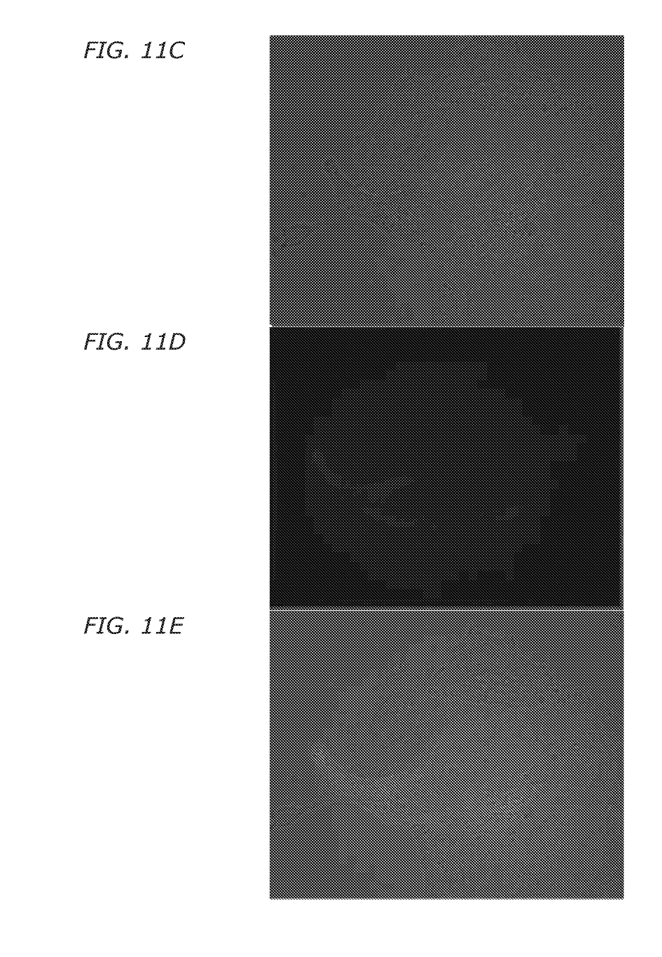

To determine if NIH45-46 G54W enters Env-expressing cells, confocal microscopy was used to examine the location of FITC-conjugated NIH45-46 G54W. 293T cells were seeded in four-well chamber plates and co-transfected with cleavage-competent Env-expressing plasmid Yu2 gp160dCT/pSVIII and HIV-1 Tat-expressing plasmid pCTAT. Two days post-transfection, 293T cells were incubated at 37° C. with 8 μg/ml of FITC-conjugated NIH45-46 G54W in serum-free medium for 4 h. After three washes with PBS, live cells were examined and imaged under a Zeiss LSM510 laser scanning confocal microscope. A FITC-conjugated human IgG was used as control. FIG. 10 demonstrates that more FITC-conjugated NIH45-46 G54W was accumulated in the activated ACH-2 cells over time. FIG. 10 shows that approximately 30% of activated ACH-2 cells were FITC-positive over a cut-off fluorescence density after 4 h incubation with FITC-conjugated NIH45-46 G54W. In order to further locate FITC-conjugated NIH45-46 G54W, live Env-expressing 293 T cells were used rather than the activated ACH-2 cells in the confocal experiment. Confocal image results (FIG. 11) were consistent with flow cytometry data. Env-expressing 293T cells showed moderate to strong florescence, which conform to the data obtained by flow cytometry. Furthermore, internalization of FITC-conjugated NIH45-46 G54W is Env-dependent since FITC-conjugated non-Env-directed antibody used as control was not taken up by the Env-expressing 293T cells (FIG. 11A-F).

### Example 2

### Killing of HIV-Infected Cells with NIH45-46 G54W Drug Conjugate

The successful demonstration of the Env-dependent internalization of NIH45-46 G54W suggests that an antibody-drug conjugate (ADC), used successfully in cancer therapy, can be used to deliver a drug (toxin) to kill HIV infected cells, thereby reducing virus reservoirs. An ADC, vc-MMAE-NIH45-46 G54W (NYBC-HC-001) was constructed and its potential to kill reactivated ACH-2 latent cells was determined. vc-MMAE-conjugated anti-HER2 and A3.01, a HIV(-) T-cell line were used as controls.

Preparation of Antibody Drug Conjugates.

Briefly, 36 mg of IgG1 (NIH45-46, NIH45-46 G54W, or control IgG1) pH 6.5-7.0 containing 5 mM EDTA was split into two 18 mg tubes and were reduced with TCEP at a 10:1 mole ratio TCEP:IgG1 for 10 min for one sample and 30 min for the other sample in a 25° C. Stability Chamber with gentle mixing using the rotary MACSMIX™. The MC-vc-PAB-MMAE linker payload was solubilized first in DMF (<5 min) and then mixed separately with each of the reduced human IgG1 samples at a molar ratio of 50:1 linker payload:reduced IgG1 to generate the conjugate. Acetonitrile was added at a 1:1 volume ratio (DMF:Acetonitrile) to give a final organic solvent concentration of 15%. The mixtures were gently mixed for 2 h using the rotary MACSMIX™ in a 25° C. stability chamber. After the 2 h incubation, residual free thiols were quenched with NEM at a ratio of 3:1 NEM:fluorescein-5-maleimide for 30 min in a 25° C. stability chamber with the rotary MACSMIX™. The conjugates were then purified/diafiltered via AMICON® 10 kDa MWCO Centrifuge Filter into DPBS pH 7.4. The conjugates were then characterized via HPLC-SEC with monitoring at wavelengths of 248 nm and 280 nm, SDS-PAGE (nonreducing), and A₂₄₈ and A₄₈₀ via SPECTRAMAX® spectrophotometer.

ACH-2 and its parental cells, A3.01 (a human T cell line before becoming latent with HIV-1), were maintained in RPMI-1640 supplemented with 10% FBS and penicillin/streptomycin. Before use in the cytotoxicity assay, the cell density of ACH-2 was adjusted to 5×10⁵/ml and TNF-α (final concentration of 10 ng/ml) was used to disrupt the latency and increase the expression of HIV-1 Env for 2 days. As a control, A3.01 cells were also treated with 10 ng/ml of TNF-α. For the cytotoxicity assay, both activated ACH-2 cells and control A3.01 cells were washed with 1×PBS twice. The cell density was then adjusted to 5×10⁴/ml and 100 μl of activated ACH-2 cells or A3.01 control cells was plated in the flat bottom 96-well plate. A 100 μl of 1:3 serially-diluted vc-MMAE-conjugated NIH45-46 G54W or vc-MMAE-conjugated anti-HER2 as control was applied in triplicate. After 3 d incubation, a CELLTITER-BLUE® cell viability assay was used to evaluate the potency of the conjugates following manufacture's instructions. The florescence density was plotted after subtracting the cell-free background and EC₅₀ was calculated using GRAPHPAD PRISM® 6.0 software.

As shown in FIG. 12, vc-MMAE-conjugated anti-HER2 had no effect on activated ACH-2 latent cells whereas vc-MMAE-NIH45-46 G54W killed the activated ACH-2 latent cells in a dosage-dependent manner. As expected, vc-MMAE-NIH45-46 G54W had no effect on the control cells. The potency of cell killing by vc-MMAE-NIH45-46 G54W (EC₅₀) is 850 ng/ml on the activated ACH-2 latent cells while up to 20 μg/ml vc-MMAE-NIH45-46 G54W showed no effect on the cell viability in A3.01 cells, which indicates that the cell killing is Env-dependent.

### Example 3

### Binding of Antibodies to Reactivated Latent HIV-Infected Cells

In order to detect reactivated HIV-infected cells, cell surface Env expression will be measured by flow cytometry. Combinations of LRAs from the same class (i.e., PKC activators) as well as a different class (i.e., PKC activators and HDAC inhibitors) are selected to target multiple sites for synergistic effects.

In preliminary studies, binding of NIH45-46 G54W to reactivated ACH-2 cells expressing the HIV-1 Env was demonstrated. An ACH-2 latency model (Clouse et al. J. Immunol. 142:431-8, 1989) is used to measure the binding of selected antibodies to the Env expressed on the latent cells after reactivation with LRA by flow cytometry. TNF-α is used as the control LRA in this latency model.

Two primary CD4+ T-cell models of HIV-1 latency are used to evaluate LRA, alone or in combination. In the first model, bulk primary CD4+ T cells from normal donors are isolated from by positive selection (MACS) from peripheral blood, and stimulated with immobilized αCD3 antibody in microplates in the presence of aCD28 antibody and rIL-2 (100 U/mL). After 3 days, the activated cells are transduced with the lentiviral vector, EB-FLV, for constitutive expression of Bcl-2, and then expanded in culture with rIL-2 for an additional 3 days. The transduced cells return to a resting state after 3-4 weeks of culture in the absence of any exogenous cytokines. At this point, viable cells are recovered and re-stimulated with immobilized αCD3 antibody+rIL-2, as before. After 10-12 days, the Bcl-2 transduced cells are infected with the HIV reporter construct pNL4-3 (a replication-competent full reporter virus (X4)) and pYK-JRCSF (R5) HIV. The infected cells are cultured with rIL-2 for 3 days, and then maintained for at least 4 weeks in the absence of exogenous cytokines. To recover latently infected CD4 cells, the GFP-negative portion of the culture is purified by flow cytometry sorting. The isolated cells having a predominant quiescent cell phenotype (G0/1a) that is representative of effector memory T cells (TEM), with expression of CD45RO+RAdim/CCR7-/CD25dim will be reactivated. Virus reactivation is measured by expression of GFP, using flow cytometry analysis.

In the second model, CD4+ T cells, with a naïve phenotype were isolated by negative selection from fresh peripheral blood and exposed to αCD3 plus aCD28 antibody-coated beads (DYNAL®, 1:1 bead:cell ratio) for 3 days, with the addition of TGF-β (10 μg/mL), αIL-4 (1 μg/mL) and αIL-12 (2 μg/mL) monoclonal antibodies. Cells are then cultured in rIL-2 for an additional 4 days to derive a differentiated “non-polarized” subset (NP), or T-helper 1 (Th1) or T-helper 2 (Th2) polarizing conditions. The NP subset is characterized by expression of CCR7, CD27, CD45RO, and the IL-7 receptor (CD127). After 7 days of activation, latent infection is established through infection with pNL4-3 (X4) and pYK-JRCSF (R5) viruses. At 3 and 5 days after reactivation, the cells are assessed for cell surface Env expression.

In both assays, all LRAs (single or combinations) are evaluated with primary CD4+ T cells from 3-4 different donors with triplicate repeats per assay. DMSO (0.5%), TNF-α, and IL-7 serve as controls. Binding of Env-specific antibodies to the reactivated cells is determined with a varied dose of the LRAs. A dose-response curve is generated and the dosage required to achieve the half-maximal (IC₅₀) Env-recognition is determined.

### Example 4

### In Vitro Killing of Latently-Infected Cells by ADCs

The ACH-2 transformed cell model and the primary CD4+ T-cell model described in Example 3 are used to assess the in vitro killing potency of the ADCs. Latent transformed cells, such as ACH-2 cells and resting primary CD4+ T cells, are first treated with an LRA (single or in combination) for 48 hours to reverse the latency and enhance expression of the cell surface HIV-1 Env. The reactivated cells (5×10⁴/ml) are then incubated with 1:3 serially diluted ADC for 3 days and the cell viability measured using the CELLTITER-BLUE® cell viability assay in accordance with the manufacturers instructions. The control ADC is vc-MMAE-conjugated anti-HER2. The florescence density is plotted by substracting the no-cell background and calculating EC₅₀. With the primary CD4+ T-cell model, additional steps will be taken to evaluate the reduction in the latent cells after administering the ADC since the normal T cells exhibit some background in the assay. Five hundred thousand (5×10⁵) reactivated cells per milliliter are incubated with the 1:3 serially diluted ADC for 11 days to reenter the latency stage—if the reactivated cells survive. After thorough wash-away of the ADC and 3 days of second-round reactivation, further FACS analysis will reveal the percentage of first-round reactivated cells that survive the ADC treatments.

### Example 5

### Evaluation of ADCs in Murine Latency Model

The ADCs are assessed for their ability to deplete latently infected cells in vivo using a humanized BLT-mouse system as an model for HIV latency. In this model, a human immune system is generated within the mouse through reconstitution with human fetal liver, thymus, and hematopoietic progenitor cells. Following HIV infection of this model, relatively high levels of cells latently infected with HIV are seen in the spleen and other organs in the presence of anti-retroviral therapy (ART). Importantly, mice in each transplant series receive fetal tissue from a single fetal tissue donor; therefore, in terms of human reconstitution, the reconstituted immune systems are genetically identical. Virus expression is induced using one or more LRAs, and then the ADCs are administered to determine the effects on the latent HIV reservoir.

Specifically, humanized BLT mice are individually bioengineered by bone marrow transplant of CD34+ hematopoietic progenitor cells into immunodeficient mice previously implanted with autologous liver and thymus tissue. Human hematopoietic cells are present in all tissues of BLT mice, including peripheral blood, primary lymphoid tissues, secondary lymphoid tissues, and mucosal tissues as previously described (Brainard et al. J. Virol. 83:7305-7321, 2009). Approximately 12 weeks following implantation, the BLT mice are infected with the HIV strain NL43 or JR-CSF. The infection is allowed to progress for 4-6 weeks, then the mice are placed on ART (raltegravir, emtricitabine, and tenofovir disoproxil fumarate) for 2-4 weeks, and monitored for plasma viral load suppression to undetectable levels by reverse transcription polymerase chain reaction (RT-PCR). The LRA(s) are then administered by IP injection.

To assess latency, 24 hours after injection of the LRA(s), ADCs are administered and 48-72 hours later, the following parameters are determined: plasma viral loads (RT-PCR), infected cell numbers (intracellular staining for Gag), HIV proviral DNA (PCR), and latently-infected cell numbers (by anti-CD3/anti-CD28 costimulation in a limiting dilution viral outgrowth assay). Control groups consist of ART-treated mice not receiving the LRA or ADC, mice receiving the LRA only, and mice receiving the ADC.

### Example 6

### Determination of the Potency of ADCs in Reducing Latent Cells in an Ex Vivo Latency Model

Peripheral blood is obtained from HIV-1-infected individuals who have been on ART for at least 6 months. In brief, resting CD4+ T cells isolated from the patient's blood are treated with LRA(s) for 18 hours and then cultured with a transformed CD4+ T-cell line (MOLT-4/CCR5) that supports robust HIV-1 replication without inducing allogenic stimulation of resting CD4+ T cells, for 14 days to permit viral outgrowth. Phorbol 12-myristate 13-acetate plus ionomycin (PMA/I) is used as a positive control for T cell activation. A quantitative RT-PCR assay specific for polyadenylated HIV-1 RNA is used for virus detection, to determine the potency of the ADCs in reducing latent cells. For comparison, an HIV-1 p24 antigen ELISA is also used.

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. An antibody-drug conjugate comprising the general formula:
A-L-D
wherein A represents a broadly neutralizing antibody specific for human immunodeficiency virus (HIV), L represents a linker, and D represents a cytotoxic moiety.

2. The antibody-drug conjugate of claim 1, wherein the antibody is a mouse antibody, a human antibody, a chimeric antibody, a humanized antibody, or an antibody fragment.

3. The antibody-drug conjugate of claim 2, wherein the antibody fragment is selected from Fab, Fab′, F(ab′)₂, and Fv fragments, diabodies, triabodies, tetrabodies, linear antibodies, single-chain antibody molecules, scFv, and scFv-Fc antibody fragments.

4. The antibody-drug conjugate of claim 1, wherein the antibody is NIH45-46 G54W.

5. The antibody-drug conjugate of claim 1, wherein the linker is a cleavable linker or a non-cleavable linker.

6. The antibody-drug conjugate of claim 5, wherein the cleavable linker is a dipeptide linker.

7. The antibody-drug conjugate of claim 6, wherein the dipeptide linker is valine-citrulline, histidine-valine, aspartic acid-valine, isoleucine-valine, asparagine-valine, tyrosine-valine, ε-N-trimethyllysine-proline, isoleucine-proline, tyrosine-aspartic acid, norvaline-aspartic acid, phenylglycine-lysine, methionine-lysine, or asparagine-lysine.

8. The antibody-drug conjugate of claim 7 wherein the dipeptide linker is valine-citrulline.

9. The antibody-drug conjugate of claim 5, wherein the non-cleavable linker is succinimidyl trans-4-(maleimidylmethyl)cyclohexane-1-carboxylate (SMMC).

10. The antibody-drug conjugate of claim 1, wherein the cytotoxic moiety is an auristatin, a maytanasine, a calicheasmicin, a duocarymycin, a PDB dimer, or an amanitin.

11. The antibody-drug conjugate of claim 10, wherein the auristatin is MMAE (N-methylvaline-valine-dolaisoleuine-dolaproine-norephedrine), MMAF (N-methylvaline-valine-dolaisoleuine-dolaproine-phenylalanine), or AF (N,N-dimethylvaline-valine-dolaisoleuine-dolaproine-phenylalanine).

12. The antibody-drug conjugate of claim 10, wherein the maytanasine is DM1 or DM4.

13. The antibody-drug conjugate of claim 1, wherein the antibody-drug conjugate is produced as a fusion protein and comprises an antibody, a dipeptide linker, and an auristatin in that order.

14. The antibody-drug conjugate of claim 13, wherein the antibody is NIH45-46 G54W, the linker is valine-citrulline, and the auristatin is MMAE.

15. (canceled)

16. (canceled)

17. A method of treating a subject infected with a human immunodeficiency virus comprising administering the antibody-drug conjugate of claim 1 to the subject.

18. The method of claim 18, wherein the method further comprises first activating latent HIV-infected cells from the latent reservoir with a latency-reversing agent.

19. A method of reducing the latent reservoir of HIV in a subject infected with HIV comprising administering the antibody-drug conjugate of claim 1 to the subject.

20. The method of claim 19, wherein the method further comprises first activating latent HIV-infected cell from the latent reservoir with a latency-reversing agent.

21. The method of claim 18, wherein the latency-reversing agent is a histone deacetylase (HDAC) inhibitor, a protein kinase C (PKC) activator, a histone methyltransferase (HMT), a DNA methyltransferase inhibitor (DNMTI), a bromodomain and extra terminal (BET) domain-containing protein inhibitor, a toll-like receptor (TLR) agonist, or a Smac (second mitochondrial-derived activator of caspases) mimetic, and combinations thereof.

22. The method of claim 20, wherein the latency-reversing agent is a histone deacetylase (HDAC) inhibitor, a protein kinase C (PKC) activator, a histone methyltransferase (HMT), a DNA methyltransferase inhibitor (DNMTI), a bromodomain and extra terminal (BET) domain-containing protein inhibitor, a toll-like receptor (TLR) agonist, or a Smac (second mitochondrial-derived activator of caspases) mimetic, and combinations thereof.


SEQUENCE LISTING (4 sequences)

SEQ ID NO: 1 - protein, 208 aa, Homo sapiens
EIVLTQSPATLSLSPGETAIISCRTSQSGSLAWYQQRPGQAPRLVIYSGSTRAAGIPDRFSGSRWGADYNLSISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRG

SEQ ID NO: 2 - protein, 455 aa, Homo sapiens
QVRLSQSGGQMKKPGESMRLSCRASGYEFLNCPINWIRLAPGRRPEWMGWLKPRWGAVNYARKFQGRVTMTRDVYSDTAFLELRSLTSDDTAVYFCTRGKYCTARDYYNWDFEHWGRGAPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 20 aa, Human immunodeficiency virus
SQNQQEKNEQELLALDKWAS

SEQ ID NO: 4 - protein, 20 aa, Human immunodeficiency virus
LWNWFDITKWLWYIKIFIMI